(19) United States
(12) Patent Application Publication
(10) Pub. No.: US 2010/0261195 A1
(43) Pub. Date: Oct. 14, 2010

(54) RAPID ANTEMORTEM DETECTION OF INFECTIOUS AGENTS

(76) Inventors: Richard Rubenstein, Staten Island, NY (US); Martin S. Piltch, Los Alamos, NM (US); Perry Clayton Gray, Los Alamos, NM (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/731,776

(22) Filed: Mar. 25, 2010

Related U.S. Application Data
(60) Provisional application No. 61/211,265, filed on Mar. 25, 2009.
(60) Provisional application No. 61/211,264, filed on Mar. 25, 2009.

(51) Int. Cl. G01N 33/53; G01N 33/50; C07K 16/00; G01N 23/00

(52) U.S. Cl. 435/7.1; 436/86; 436/501; 530/388.1; 436/63; 436/57

(57) ABSTRACT

Methods for detection of the presence or absence of PrP^{Sc} in a biological sample suspected of having them comprising the steps of concentrating the PrP^{Sc} as may be present in the sample by substantially separating the PrP^{Sc} from the sample matrix; labeling the concentrated PrP^{Sc} with at least one molecular label to produce labeled PrP^{Sc}; and detecting the labeled PrP^{Sc} on an instrument capable of detecting an attomole quantity of labeled PrP^{Sc}, and wherein the duration of time between concentrating the PrP^{Sc} and analyzing the labeled PrP^{Sc} is about 48 hours or less.

### CROSS REFERENCE TO RELATED APPLICATION

This application claims the benefit of priority of U.S. Provisional Patent Application 61/211,265, filed Mar. 25, 2009, incorporated by reference herein in its entirety. This application claims the benefit of priority of U.S. Provisional Patent Application 61/211,264, filed Mar. 25, 2009, incorporated by reference herein in its entirety.

### STATEMENT OF FEDERAL RIGHTS

These inventions were made with government support under Contract No. DE-AC52-06 NA 25396, awarded by the U.S. Department of Energy. The government has certain rights in the inventions. These inventions further were made with support from grant number DAMD17-03-1-0368, awarded by the Army Medical Research and Materiel Command, and grant number HL063837, awarded by the National Heart Lung Blood Institute.

### FIELD OF THE INVENTIONS

The present inventions relate to methods of rapid, antemortem detection of trace amounts of biological and chemical products, exemplary among those are the conformationally altered form of cellular prion protein in biological samples.

### BACKGROUND

The transmissible spongiform encephalopathies (TSEs), or prion diseases, are infectious neurodegenerative diseases of mammals that include bovine spongiform encephalopathy (“mad cow” disease), chronic wasting disease of deer and elk, scrapie in sheep, and Creutzfeldt-Jakob disease (CJD) in humans. TSEs may be passed from host to host by ingestion of infected tissues or blood transfusions. Clinical symptoms of TSEs include loss of movement coordination and dementia in humans. They have incubation periods of months to years, but after the appearance of clinical signs, they are rapidly progressive, untreatable and invariably fatal. Attempts at TSE risk reduction have led to significant changes in the production and trade of agricultural goods, medicines, cosmetics, blood and tissue donations, and biotechnology products.

TSEs are associated with the conversion of host-encoded, cellular prion protein (PrP^{C}) into a conformationally altered form (PrP^{Sc}). Post-mortem neuropathological examination of brain tissue from an animal or human has remained the ‘gold standard’ of TSE diagnosis and typically reveals astrocytosis and spongiform changes, sometimes accompanied by the formation of PrP^{Sc}-containing amyloid deposits. It is very specific but less sensitive than other techniques (Wells and Wilesmith, 1995; Gavier-Widen et al., 2005). Although the sensitivity of microscopic observation can be increased by immunohistochemical techniques that use antibodies specific to PrP to detect accumulation of PrP^{Sc} in amyloid deposits (van Keulen et al., 1995; 1996), these methods are ill-suited to rapid, routine analysis. An additional concern is that laboratory diagnosis of TSEs is complicated by the uneven distribution of TSE associated molecules in body tissues, with highest concentrations consistently found in nervous system tissues and very low concentrations in easily accessible body fluids such as blood or urine.

PrP^{Sc} has distinct physiochemical and biochemical properties such as aggregation, insolubility, protease digestion resistance, and a β-sheet-rich secondary structure. One such altered property of PrP^{Sc}, namely, partial resistance to protease digestion, forms the basis of the majority of diagnostic biochemical tests. To differentiate between PrP^{C} and PrP^{Sc}, the sample is typically pretreated with proteinase K (PK). Since PrP^{Sc} is partially digestion resistant and PrP^{C} is easily digested by PK, pretreatment results in elimination or reduction of interference from PrP^{C}, and in a sample that is rich in PrP^{Sc} as compared to PrP^{C}. However, it has been suggested by others that the majority of PrP^{Sc} in the brains of patients who died from CJD is a PK-sensitive version of PrP^{Sc} (sPrP^{Sc}), making the use of PK treatment in an antemortem assay, where PrP^{Sc} concentrations are very low, impractical. The development of diagnostic assays that do not require proteolytic treatment of samples would eliminate the issues associated with proteolytic digestion and reduced assay sensitivity.

Current PrP^{Sc} detection methods are time-consuming and employ post-mortem analysis after suspicious animals manifest one or more symptoms of the disease. Current diagnostic methods are based mainly on detection of physicochemical differences between PrP^{C} and PrP^{Sc} which, to date, are the only reliable markers for TSEs. For example, the most widely used diagnostic tests exploit the relative protease resistance of PrP^{Sc} in brain samples to discriminate between PrP^{C} and PrP^{Sc}, in combination with antibody-based detection of the PK-resistant portion of PrP^{Sc}. It has as yet not been possible to detect prion diseases by using conventional methods such as polymerase chain reaction, serology or cell culture assays. An agent-specific nucleic acid has not yet been identified, and the infected host does not elicit an antibody response.

Antibody-antigen binding events of PrP^{Sc} to three antibodies discussed herein (8E9, 11F12, and 5D6) have been characterized in an electronically published Oct. 31, 2008 publication by Chang, et al., PrP Antibody Binding-Induced Epitope Modulation Evokes Immunocooperativity, 205 J. Neuroimmunol., 94, 94-100, the contents of which are hereby incorporated herein in its entirety. These antibodies interact with different epitopes on PrP^{Sc}. Monoclonal antibody (Mab) 8E9 binds in the region of amino acids 155-200 of PrP^{Sc}. Mab 11F12 binds in the region of amino acids 93-122 of PrP^{Sc}. Mab 5D6 binds to an undefined conformational epitope of PrP^{Sc}. A conformational epitope does not bind to a specific continuous sequence of amino acids. Rather it binds to a region of the protein's structure that can include amino acid residues from several, disconnected areas of the amino acid primary structure.

Capture enzyme-linked immunosorbent assays (ELISAs) were performed using these three antibodies. Only using Mab 11F12 as the capture reagent and using the biotinylated monoclonal antibody 5D6 as the detector was successful in binding to and identifying PrP^{Sc}. Only this combination of antibodies in this order provided the same results in 263K-infected hamsters, scrapie sheep or CWD-affected deer. Detection was further enhanced using heat and or sodium dodecylsulfate (SDS) denaturation. It is believed that this increased detection is due to antibody induced epitope unmasking in PrP^{Sc}. In essence, binding of one antibody (Mab 11F12) to PrP^{Sc} unmasks an epitope in some way to allow a second antibody (Mab 5D6) to bind better. It is not known whether this occurs through PrP conformational alterations, refolding of PrP^{C} into PrP^{Sc} and/or changes in the PK-resistant or sPrP^{Sc} forms to make them more accessible to additional antibody binding.

Surround optical fiber immunoassay (SOFIA) was also disclosed in an electronically published Feb. 27, 2009 publication by Chang et. al., Surround Optical Fiber Immunoassay (SOFIA): An Ultra-Sensitive Assay for Prion Protein Detection, 159 Journal of Virological Methods, 15, 15-22. SOFIA combines the specificity inherent in Mabs for antigen capture with the sensitivity of surround optical detection technology. To detect extremely low signal levels, a low noise, photo-voltaic diode was used as the detector for the system. SOFIA utilizes a laser illuminating a micro-capillary holding the sample. Then, the light collected from the sample is directed to transfer optics from optical fibers. Next, the light is optically filtered for detection, which is performed as a current measurement and amplified against noise by a digital signal processing lock-in amplifier. The results are displayed on a computer and stored on computer software designed for data acquisition.

Rhodamine Red was detectable by SOFIA to a concentration of 0.1 attograms (ag). Thus, SOFIA shown there had a detection limit of approximately 10 ag of PrP^{Sc} from non-PK treated hamster brain, and extrapolating, about 1 femtogram of PrP^{Sc} from sheep and deer brain material. However, assuming equal antibody reactivity, western blotting indicated that there is at least 10-100 fold more PrP^{Sc} in hamster brains than in sheep and deer brain material on a gram equivalent basis suggesting that detection of the protein in the latter two species could be in the range of 10-100 ag or better.

The laboratory technique of protein misfolding cyclic amplification (PMCA) has been reported to support the specific reproducible conversion of PrP^{C} to PrP^{Sc} resulting in the amplification of minute quantities of PrP^{Sc}. Although the CWD infectious agent has been detected in saliva, blood, urine and feces, the direct immunodetection of PrP^{Sc} from this material has been unsuccessful (Haley et al., 2009a, b). Furthermore, the successful detection of the CWD infectious agent for some of this material required bioassays of the serial PMCA (sPMCA) products (Mathiason et al., 2006, 2009; Haley et al., 2009a, b; Tamguney et al., 2009). To facilitate preclinical detection of TSEs in peripheral tissues, notably blood, the target PrP^{Sc} in a sample can be amplified by means of PMCA (Saborio et al., 2001). PMCA has been reported to increase the sensitivity of the detection of PrP^{Sc} from brains of experimentally scrapie-infected rodents (Saborio et al., 2001; Deleault et al., 2003; Bieschke et al., 2004), cattle and sheep naturally infected with bovine spongiform encephalopathy and scrapie, respectively (Soto et al., 2005), and more recently from humans with Creutzfeldt-Jakob disease (Jones et al., 2007) and deer with chronic wasting disease (Kurt et al., 2007). Furthermore, PMCA has been reported to detect PrP^{Sc} in sheep and hamster blood, both at terminal stages of disease and in pre-symptomatic animals (Castilla et al., 2005a, b; Saa et al., 2006; Murayama et al., 2007; Thorne and Terry, 2008) and in urine and cerebrospinal fluid (Atarashi et al., 2007, 2008; Murayama et al., 2007) making this technology a useful diagnostic tool. However, to date PMCA is hindered by the need for many rounds of cycling in order to visualize the final product by immunoblotting. In fact, performing many rounds of PMCA can lead to false-positive results. By 192 cycles, control blood samples showed the spontaneous conversion of PrP^{C} to PrP^{Sc}, thus making this technique somewhat inadequate for diagnostic purposes. PMCA has great potential, but is hampered by various fundamental and technical difficulties including the length of time necessary to achieve optimal sensitivity (approximately 3 weeks).

The current dogma is that PrP^{Sc} directly correlates with infectivity and their accumulation in the brain causes neuropathology and clinical disease. It is also assumed that the rate and pattern of PrP^{Sc} accumulation, and, therefore, the rate of formation of neuropathology, determines the incubation periods of the disease (Prusiner et al., 1990; Carlson et al., 1994). However, it has also been shown that in the CNS and contrary to expectation, overall accumulation of PrP^{Sc} and infectivity to a high level can be present in asymptomatic mice (Bueler et al., 1994). Additional studies on naturally and experimentally infected sheep (Madec et al., 2004; Bulgin et al., 2006) have also demonstrated inconsistencies between the levels of PrP^{Sc}, IHC staining topology, extent of histological lesions and clinical disease.

To improve food safety it would be highly beneficial to screen all the animals for prion disease using antemortem, pre-clinical testing, i.e., testing prior to presentation of symptoms. However, PrP^{Sc} levels are very low in pre-symptomatic hosts. In addition, PrP^{Sc}s are generally unevenly distributed in body tissues, with highest concentrations consistently found in nervous system tissues and very low concentrations in easily accessible body fluids such as blood or urine. Therefore, any such test would be required to detect extremely small amounts of PrP and would have to differentiate PrP^{C} and PrP^{Sc}.

The ability to secure early diagnosis is vital for therapeutic interventions to be of real value. With respect to animals destined for the human food chain and blood and tissue donors, prion agents must be detectable well before the appearance of any clinical symptoms. Thus, there is a continuing need for more sensitive methods of prion detection.

### SUMMARY

The conformationally altered form of PrP^{C} is PrP^{Sc}. Some groups believe that PrP^{Sc} is the infectious agent (prion agent) in TSEs, while other groups do not. PrP^{Sc} could be a neuropathological product of the disease process, a component of the infectious agent, the infectious agent itself or something else altogether. Regardless of what its actual function in the disease state is, what is clear is that PrP^{Sc} is specifically associated with the disease process and detection of it indicates infection with the agent that causes prion diseases.

The present inventions provide, among other things, methods to diagnose prion diseases by detection of PrP^{Sc} in a biological sample. This biological sample can be brain tissue, nerve tissue, blood, urine, lymphatic fluid, cerebrospinal fluid, or a combination thereof. Absence of PrP^{Sc} indicates no infection with the infectious agent up to the detection limits of the methods. Detection of a presence of PrP^{Sc} indicates infection with the infectious agent associated with prion disease. Infection with the prion agent may be detected in both presymptomatic and symptomatic stages of disease progression.

These and other improvements have been achieved with SOFIA, a laser-based immunoassay which has been developed for the detection of PrP^{Sc} (Chang et al., 2009). SOFIA's sensitivity and specificity (Chang et al., 2009) eliminates the need for PK digestion to distinguish between the normal and abnormal PrP isoforms. Further, the detection of PrP^{Sc} in blood plasma has now been addressed by limited PMCA followed by SOFIA. Because of the sensitivity of SOFIA, PMCA cycles can be reduced, thus decreasing the chances of spontaneous PrP^{Sc} formation and the detection of falsely positive samples.

The present inventions meet the aforementioned needs of increased sensitivity in the detection of prion diseases in both presymptomatic and symptomatic TSE infected animals, including humans, by providing methods of analysis using highly sensitive instrumentation, which requires less sample preparation than previously described methods, in combination with recently developed Mabs against PrP. The methods of the present inventions provide sensitivity levels sufficient to detect PrP^{Sc} in brain tissue. When coupled with limited sPMCA, the methods of the present inventions provide sensitivity levels sufficient to detect PrP^{Sc} in blood plasma, tissue and other fluids collected antemortem. The time between sample collection and analysis can be less than 24 hrs for brain material The methods combine the specificity of the Mabs for antigen capture and concentration with the sensitivity of a surround optical fiber detection technology. In contrast to previously described methods for detection of PrP^{Sc} in brain homogenates, these techniques, when used to study brain homogenates, does not utilize seeded polymerization, amplification, or enzymatic digestion (for example, by proteinase K, or “PK”). This is important in that previous reports have indicated the existence of PrP^{Sc} isoforms with varied PK sensitivity, which decreases reliability of the assay. The sensitivity of this assay makes it suitable as a platform for rapid prion detection assay in biological fluids. In addition to prion diseases, the method may provide a means for rapid, high-throughput testing for a wide spectrum of infections and disorders.

While it was found that about 40 cycles of sPMCA combined with immunoprecipitation was inadequate for PrP^{Sc} detection in plasma by ELISA or western blotting, the PrP^{Sc} has also been found to be readily measured by SOFIA methods. In accordance with this invention the limited number of cycles necessary for the present assay platform virtually eliminates the possibility of obtaining PMCA-related false positive results such as those previously reported (Thorne and Terry, 2008).

The following represent non-limiting embodiments of the present invention. According to a first embodiment, methods for detection of the presence or absence of PrP^{Sc} in a biological sample suspected of having them are disclosed comprising the steps of concentrating PrP^{Sc} as may be present in the sample by substantially separating the PrP^{Sc} from sample matrix; labeling concentrated PrP^{Sc} with at least one molecular label to produce labeled PrP^{Sc}; and detecting the labeled PrP^{Sc} on analytical instrumentation.

According to a second embodiment of the present invention, methods for detection of the presence or absence of PrP^{Sc} in a biological sample suspected of having them are disclosed comprising the steps of concentrating PrP^{Sc} as may be present in the sample by substantially separating the PrP^{Sc} from sample matrix; labeling concentrated PrP^{Sc} with at least one molecular label to produce labeled PrP^{Sc}; and detecting the labeled PrP^{Sc} on analytical instrumentation. In this embodiment, the PrP^{Sc} are undigested.

According to a third embodiment of the present invention, methods for detection of the presence or absence of PrP^{Sc} in a biological sample suspected of having them are disclosed comprising the steps of concentrating PrP^{Sc} as may be present in the sample by substantially separating the PrP^{Sc} from sample matrix; labeling concentrated PrP^{Sc} with at least one molecular label to produce labeled PrP^{Sc}; and detecting the labeled PrP^{Sc} on analytical instrumentation. The duration of time between concentrating the PrP^{Sc} and analyzing the labeled PrP^{Sc} is preferably about 48 hours or less.

According to a further embodiment of the present invention, methods for detection of the presence or absence of PrP^{Sc} in a biological sample suspected of having them are disclosed comprising the steps of amplifying PrP^{Sc} in the sample by sPMCA; concentrating PrP^{Sc} as may be present in the sample by substantially separating the PrP^{Sc} from sample matrix; labeling concentrated PrP^{Sc} with at least one molecular label to produce labeled PrP^{Sc}; and detecting the labeled PrP^{Sc} on analytical instrumentation.

According to a further embodiment of the present invention, methods for detection of the presence or absence of PrP^{Sc} in a biological sample suspected of having them are disclosed comprising the steps of amplifying PrP^{Sc} in the sample by sPMCA; concentrating PrP^{Sc} as may be present in the sample by substantially separating the PrP^{Sc} from sample matrix; labeling concentrated PrP^{Sc} with at least one molecular label to produce labeled PrP^{Sc}; and detecting the labeled PrP^{Sc} on analytical instrumentation. In this embodiment, the biological sample is brain tissue, nerve tissue, blood, urine, lymphatic fluid, cerebrospinal fluid or a combination thereof.

### BRIEF DESCRIPTION OF THE DRAWINGS

FIG. 1 is a schematic representation showing one embodiment of instrumentation suitable for analysis of PrP^{Sc} according to some method of the present invention.

FIG. 2 is a schematic representation showing a side view of one embodiment of an end port assembly of instrumentation suitable for analysis of PrP^{Sc} according to some methods of the present invention.

FIG. 3 is a schematic representation of one embodiment of a sample container of instrumentation suitable for analysis of PrP^{Sc} according to methods of the present invention.

FIG. 4 is a schematic representation of the sample container of FIG. 3, as viewed from one side.

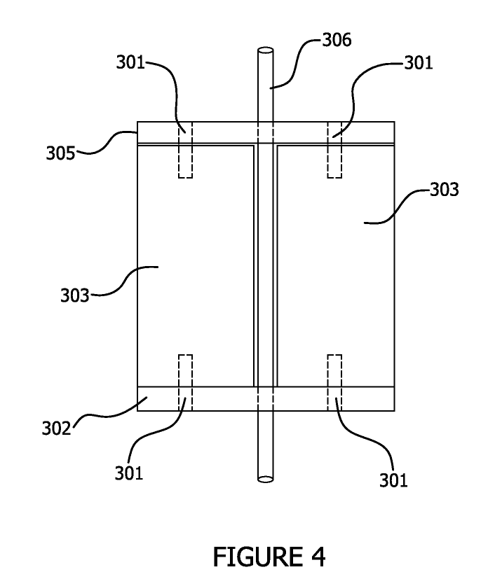

FIG. 5 is a schematic representation of the sample container of FIG. 3, as viewed from the top.

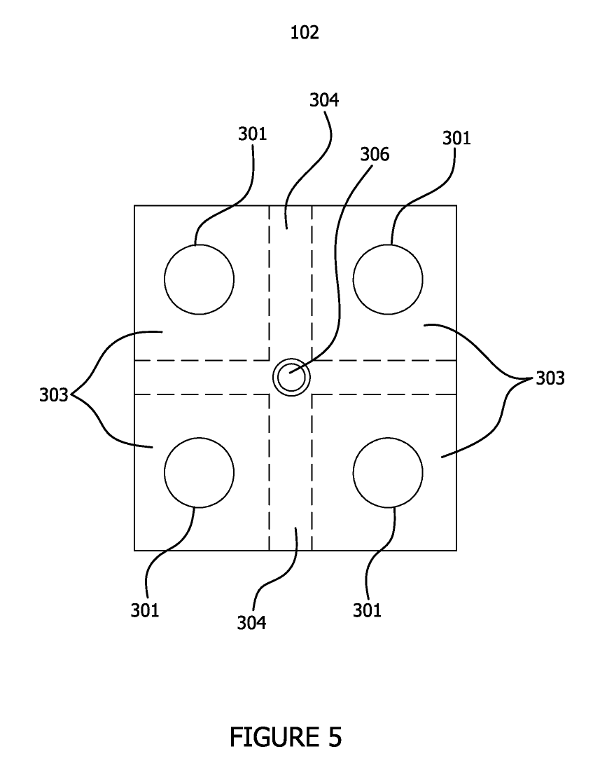

FIG. 6 depicts a western blot analysis of untreated and PK treated total brain lysates from 263K-infected hamsters (H), scrapie-infected sheep (S) and CWD-infected deer (D) using Mabs 08-1/5D6 (A), 08-1/11F12 (B), and 08-1/8E9 (C).

FIG. 7 depicts antibody binding measured colorimetrically at OD₄₀₅. Capture ELISA assay using Mabs 11F12 as the capture reagent and biotinylated 5D6 as the detection reagent. Brain tissue homogenates from normal and infected hamsters, sheep and deer. The assay was performed on non-PK and PK-treated brain lysates.

FIG. 8 depicts a western blot analysis of non-PK treated brain homogenates following capture ELISA. The capture ELISA was carried out on normal sheep (NS), scrapie-infected sheep (SS), normal deer (ND), CWD-infected deer (CWD), normal hamster (NH) and 263-K-infected hamsters (263K) under the same conditions as described in FIG. 7 using a non-biotinylated detection reagent. Immunostaining was carried out using Mab 8E9.

FIG. 9 depicts a comparison of reversing the capture and detection reagents in the capture ELISA using brain lysates from uninfected and infected hamsters, sheep and deer. Studies using 5D6 as the capture reagent and 11F12 as the biotinylated detection reagent (5D6/Biotin 11F12) are compared to using 11F12 as the capture reagent and 5D6 as the biotinylated detection reagent (11F12/Biotin 5D6).

FIG. 10 depicts data obtained on the instrument of FIG. 1, showing dilutions of Rhodamine Red (▪) and relative signal intensities from rPrP (recombinant PrP) from mouse (*), hamster (♦), sheep (▾) and deer ().

FIG. 11 depicts PrP detection by the instrument of FIG. 1 in PK-treated and untreated normal (open bar) and infected (solid bar) brain homogenates from infected hamsters, sheep and deer. The x-axis numbers represent the degree of 10-fold serial dilutions of the original samples. For example, −10 for hamster indicates that the sample has been diluted by a factor of 1×10⁻¹⁰.

FIG. 12 depicts a western blot analysis of PrP Following Mab 8E9 immunoprecipitation.

FIG. 13 depicts the results of a capture ELISA analysis of Mab 8E9 immunoprecipitation of PrP.

FIG. 14 depicts a western blot of PrP^{Sc} following sPMCA.

FIG. 15 depicts immunohistochemistry of scrapie sheep third eyelid lymphoid tissue. PrP^{Sc} immunohistostaining (red) can be seen inside follicles.

FIG. 16 depicts PrP^{Sc} detection in sheep scrapie blood samples using SOFIA with and without sPMCA.

FIG. 17 depicts PrP^{Sc} detection in CWD blood samples using SOFIA with and without sPMCA.

### DETAILED DESCRIPTION OF THE INVENTION

“PrP^{Sc}” will be understood to mean the conformationally altered form of PrP^{C}. PrP^{Sc} is specifically associated with the disease process and detection of it indicates infection with the agent that causes prion diseases. (TSE's) will be understood to include, but are not limited to, the human diseases Creutzfeldt-Jakob disease (CJD), Gerstmann-Sträussler-Scheinker syndrome (GSS), fatal familial insomnia (FFI), and kuru, as well as the animal forms of the disease: bovine spongiform encephalopathy (BSE, commonly known as mad cow disease), chronic wasting disease (CWD) (in elk and deer), and scrapie (in sheep). It is to be understood that “proteinaceous” means that the prion may comprise proteins as well as other biochemical entities, and thus is not intended to imply that the prion is comprised solely of protein.

“Substantially separating,” as used in the context of concentrating the PrP^{Sc}, is understood to mean that any sample matrix or non-PrP^{Sc} material that remains in the sample is insufficient to be detected, or to interfere with detection, by the method described herein.

“Labeled PrP^{Sc}” will be understood to mean PrP^{Sc} to which a fluorescent label has been covalently or non-covalently attached. Preferably, one fluorescent label is attached to a single PrP^{Sc} molecule.

“Capable of detecting” means that an instrument produces a signal that is significantly higher than the background noise signal of the instrument when a sample containing no labeled PrP^{Sc} is analyzed. Although the particular sample may contain greater than attomole quantities, it is understood were the sample to be diluted to approximately 0.1 attomole per milliliter of sample of labeled PrP^{Sc} that, upon analysis, the instrument would produce a reproducible and statistically significant signal.

“Attomole quantities,” means from 0.1 attomole to 1 femtomole.

“Antemortem” is understood to mean prior to death of the organism from which the sample is collected.

“Preclinically” or “presymptomatically” is understood to mean that the sample is collected from an organism that does not exhibit symptoms of a prion disease.

“Seeded polymerization” is understood to mean inducing conversion of PrP^{C} to PrP^{Sc} that has higher beta-pleated sheet content and that is protease resistant.

“Enzymatic digestion” is understood to mean breakdown of proteins by proteases, intentionally introduced into the sample, which induce selective cleavage between specific amino acids. “Enzymatic digestion” is understood not to include autodigestion or digestion due to enzymes naturally present in the sample. “Undigested,” as used herein, is understood to mean that PrP^{Sc}s are at no time during the sample preparation or analysis subjected to enzymatic digestion.

The methods of the present invention comprise the step of obtaining a sample that may or may not contain the abnormal isoform of PrP (PrP^{Sc}), for example, from an animal or human of which it is desired to determine whether infection has occurred. If the sample is from an infected organism, the sample comprises PrP^{Sc} and a sample matrix, understood to include non-PrP^{Sc} components such as cells, cellular components, biomolecules, non-PrP^{Sc} proteins, etc. The sample may be collected from and comprise nervous tissue, blood, urine, lymphatic fluid, cerebrospinal fluid, other bodily fluids, and combinations thereof.

Once collected, the PrP^{Sc} are at least semi-purified, or concentrated, by separating the PrP^{Sc} of interest from the sample matrix. The concentration may occur by a variety of means that would be known to one of skill in the art, including but not limited to the use of molecular antibodies, immunoprecipitation, magnetic beads, antibody capture on a plastic surface, methods utilizing sodium phosphotungstate, methanol, and combinations thereof. In one embodiment, the concentration occurs by using monoclonal antibodies. In SOFIA, several PrP-specific Mabs, which have recently been described to have a synergistic effect when used together in a capture ELISA were used. (Chang et al., PrP Antibody Binding-induced Epitope Modulation Evokes Immunocooperativity, J. of Immunology, v. 205, issue 1-2, pp. 94-100 (2008)).

The concentration further may occur by means of the technique described in Kim et al., 2005, incorporated herein by reference, which is an immunoprecipitation-based capture assay using a dye-labeled anti-PrP Mab along with a second biotinylated anti-PrP Mab and streptavidin-conjugated magnetic beads. Variations of this technique included dye-labeled anti-PrP Mabs with a second PrP Mab conjugated directly to magnetic beads.

The concentrated sample may comprise at least 0.1 attomole of PrP^{Sc}, alternatively at least 200 attomole, alternatively from about 0.1 attomole to about 1.0 nanomole, alternatively from about 0.1 attomole to about 1 femtomole, and alternatively from about 0.4 to about 1.0 attomole of PrP^{Sc}.

The PrP^{Sc} in the concentrated sample may be labeled with one or more fluorescent molecules to produce labeled PrP^{Sc}. The labeling may occur by a variety of methods known to one of skill in the art, including but not limited to fluorescent labeling, phosphorescent labeling, radioisotope labeling, biotinylation, and other means of labeling that would be understood by one of skill in the art. In one embodiment, the labeling is fluorescent labeling, and the fluorescent label is Rhodamine Red.

In an alternative embodiment, the PrP^{Sc} may be detected by means other than fluorescence, including but not limited to phosphorescence, absorption of infrared, visible and ultraviolet wavelengths, and by other spectroscopic means that would be understood by one of skill in the art.

In one embodiment, the concentrated sample is then analyzed on a suitable analytical instrument which is capable of sensitive and rapid detection of the PrP^{Sc}. In one embodiment, the instrument is capable of detection of attomole quantities of labeled PrP^{Sc}. In one embodiment, the time comprising the steps of concentrating the PrP^{Sc}, labeling the PrP^{Sc} and detection is 48 hours or less, alternatively 24 hours or less, and alternatively is 12 hours or less, and alternatively is 3 hours or less.

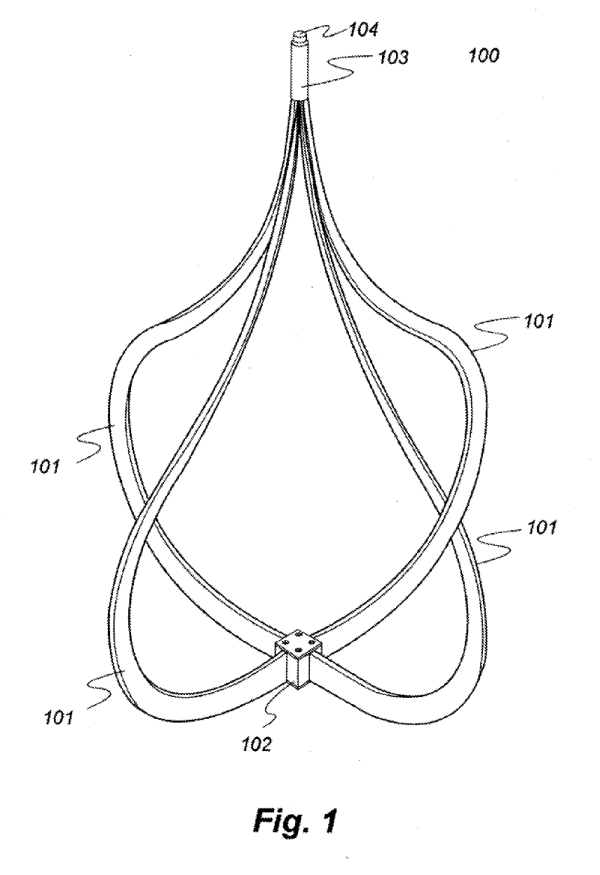

In one embodiment, instruments such as those described in U.S. patent application Ser. No. 11/634,546, filed on Dec. 7, 2005, and incorporated herein by reference in its entirety may be employed for the purposes of this invention. An alternative embodiment of the system 100 is depicted in FIG. 1. In this embodiment, four linear arrays 101 extend from a sample holder 102, which houses an elongated, transparent sample container 306, to an end port 103. The distal end of the endport 104 is inserted into an end port assembly 200. The linear arrays comprise a plurality of optical fibers having a first end and a second end, the plurality of optical fibers optionally surrounded by a protective and/or insulating sheath. The number of fibers may vary, and in one embodiment is from about 10 to about 100, alternatively is from about 25 to about 75, and alternatively is about 50. The number of linear arrays may vary, and is at least two. The maximum number of linear arrays is dependent upon the size of the sample holder in that the sample holder must be large enough to afford sufficient space for the first ends of the optical fibers to surround and be in close proximity (e.g., from about 1 mm to about 1 cm) to a sample container. In one embodiment, the number of linear arrays is from 2 to 10, alternatively is from about 4 to 6, and alternatively is 4. In one embodiment, the linear arrays are disposed in a planar array, wherein the adjacent linear arrays are oriented equidistantly from one another and surrounding the sample holder. When the number of linear arrays is four, the adjacent linear arrays are oriented at 90 degree angles with respect to each other. The length of the linear array may vary widely and is dependent upon the number and nature of the optical fibers. The length must be sufficient to allow bundling of the optical fibers from each linear array without compromising the integrity of the optical fibers. In principle, there is no upper limit on the length of the optical fibers, which would allow for a sample to be located remotely from the diagnostic equipment used to analyze the sample.

The first ends of the optical fibers may be disposed in a substantially linear manner along the length of the container comprising the sample. The second ends of the optical fibers are bundled together to form a single end port. In other words, a given length of the second ends of the fibers from each linear array are intermingled to form a single bundle. Preferably, the second ends of the fibers from each linear array are randomly interspersed within the bundle. The plurality of optical fibers receives the signal emitted from the analyte of interest and transmits the signal from the first ends of the fibers to the end port comprising the second ends of the fibers. The fibers have a high numerical aperture (NA), which corresponds to sine θ/2, where θ is the angle of accepted incident light (optical acceptance angle). In this embodiment, the NA may range from about 0.20 to about 0.25 and the optical acceptance angle of from about 20 degrees to about 45 degrees. The optical acceptance angle is chosen such that substantially all of the emitted signal may be intercepted by the plurality of fibers. This ensures optimum collection efficiency of the signal from dilute analytes, such as PrP^{Sc}.

In one embodiment, the optical fibers comprise fused silica. The fibers may have a diameter of from about 50 micrometers to about 400 micrometers.

The bundling of the optical fibers from each linear array offers several advantages. Rather than separate detectors for each linear array being required, a single detector may be used. For a system comprising four linear arrays, this results in a detection area having one-quarter the size of four individual detectors. The background noise thus is dramatically decreased, which in turn increases the signal to noise ratio and thus lowers the limit of detection. In one embodiment, the size of the detector is from about 0.5 mm×0.5 mm to about 1 mm×1 mm. The limit of detection of the system of this embodiment is at least 0.1 attomole of analyte, alternatively is at least 200 attomole, alternatively is from about 0.1 attomole to about 1.0 micromole, alternatively is from about 0.1 attomole to about 1 nanomole, and alternatively is from about 0.4 to about 1.0 attomole of analyte. Alternatively, in this embodiment, the limit of detection of the system is at least 0.1 attogram of analyte, and alternatively is at least 10 attogram of analyte.

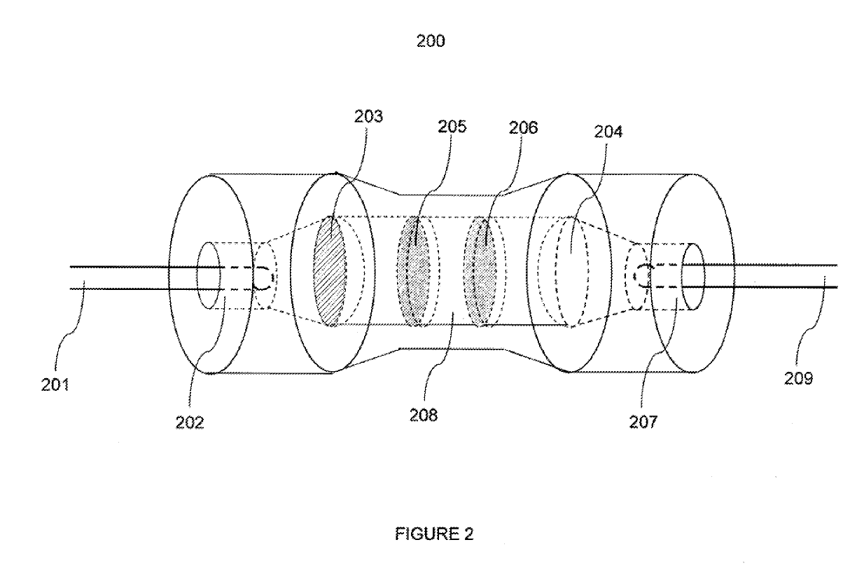

FIG. 2 depicts one embodiment of an endport assembly of this embodiment. The distal end of the single endport 104 comprising the bundled optical fibers is inserted into the entrance 202 of endport assembly 200. The signal is transmitted by the optical fibers through the endport assembly 200 to the exit 207, and is then transmitted to outgoing optical fiber 208 which in turn is in contact with a detector. Outgoing optical fiber 208 may have a diameter of from about 300 microns to about 500 microns, and preferably is about 400 microns. Therefore, the end port assembly optically couples the single end port to the detector. The endport assembly may comprise a first lens 203, which serves to collimate the incident signal. The endport assembly further may comprise a second lens 204, which serves to focus the outgoing signal to a NA suitable for outgoing optical fiber 208. The endport assembly further may comprise at least one notch filter 205 and at least one bandpass filter 206.

Non-limiting examples of suitable detectors include photo-diode detectors, photo-multipliers, charge-coupled devices, a photon-counting apparatus, optical spectrometers, and any combination thereof.

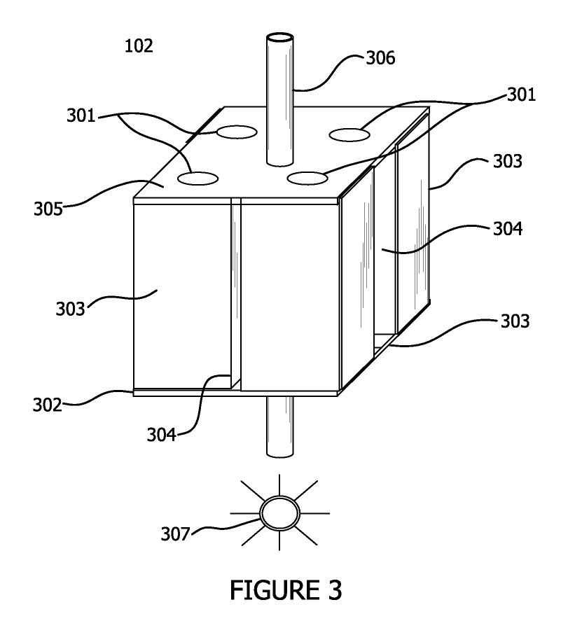

FIG. 3 depicts one embodiment of a suitable sample holder 102 of this embodiment. Spacers 303 are positioned such as to provide a space for an elongated, transparent container 306 to pass through the sample holder 300. In one embodiment, the sample holder 300 is a capillary, and may be made of glass, quartz, or any other suitable material that would be known to one of skill in the art. By way of example only, the capillary may hold 100 microliters of fluid. Spacers 303 further are positioned to provide a slot 304, or space, for the first ends of the optical fibers to surround and be in close proximity to the transparent container. Spacers 302 are held in place by top end plate 305 and bottom end plate 302, both of which are attached to the spacers 303 by a means for fastening 301, such as a screw.

The emitted signal that is captured is converted to an electrical signal by photo-detector and transmitted to an analyzer (not shown), which receives the electrical signal and analyzes the sample for the presence of the analyte. Examples of analyzers would be well-understood by those of skill in the art. The analyzer may include a lock-in amplifier, which enables phase sensitive detection of the electrical signal, or any other means known in the art for analyzing electric signals generated by the different types of photo-detectors described herein.

The apparatus developed for these assays may be optimized for the collection of the light from the reporter molecule. The dyes currently used in fluorescence based assays have quantum efficiencies near or above 90%. In one embodiment, the dye is Rhodamine Red X (Invitrogen Corp., Carlsbad Calif.). In addition, the transconductance pre-amplifier and the lock-in detector settings are optimized to facilitate low signal/low noise detection. First, an appropriate modulation frequency is chosen for the optical chopper, which should be incommensurate with the line-frequency or other electrical sources of noise in the environment. In addition, line filtering by a lock-in amplifier should be employed. In one embodiment, the modulation frequency is 753 Hz, and the lock-in amplifier is set to filter at 60 Hz and 120 Hz. The sensitivity for the transconductance pre-amplifier was chosen based on expected signal level, and to maximize the pre-amplifier's input impedance, and in one embodiment is set to 1 nA/V. In one embodiment, the bandpass filter is centered on the chopper frequency, which is e.g. 753 Hz.

### EXAMPLES

### 1. Collection of Tissue Samples

The procurement and propagation of the hamster-adapted 263K scrapie strain was as described Chang, B. et al., “PrP Antibody Binding-Induced Epitope Modulation Evokes Immunocooperativity,” J. Neuroimmunol. v. 205, issue 1-2, pp. 94-100 (2008)). Brains from sheep infected with scrapie and white-tailed deer infected with CWD were harvested at the time of clinical disease and frozen at −80° C. Brains from uninfected animals were similarly harvested and frozen. The coding region of the full-length deer, hamster, mouse and sheep PrP was cloned into a pET-23 vector to produce a tag-free protein (rPrP) as described in D. R. Brown et al., “Normal prion protein has an activity like that of superoxide dismutase,” Biochem J. vol. 344 pp. 1-5 (1999). Expression and purification was substantially identical to procedures C. E. Jones et al., “Preferential Cu²⁺ coordination by His⁹⁶ and His¹¹¹ induces β-sheet formation in the unstructured amyloidogenic region of the prion protein,” J. Biol. Chem. 279, pp. 32018-32027 (2004).

Experimental oral infections used a 20% scrapie sheep brain homogenate (derived from a composite of 7 scrapie brains from clinically and immunohistochemically positive animals) prepared in phosphate-buffered saline (PBS). All uninfected animals were housed in a separate scrapie-free facility. Clinical signs of sheep scrapie included: fine head tremors progressing to body trembling, wool loss from rubbing, nibbling at extremities, hypersensitivity and gait abnormalities.

Genotyping of the sheep was performed commercially (Gene Check, Inc., Greeley, Colo.).

For IHC, formalin fixed third eyelid tissues were washed for 15 min in water and soak in 99% formic acid for 1 hr. After a 3 hr water wash, the tissues were paraffinized in a Microm STP 120, and cut at 4 microns for mounting. The slides were allowed to dry for at least 24 hours, deparaffinized and then immunostained using the Ventana (Ventana Medical Systems Inc., Oro Valley, Ariz.) proprietary reagents (prion enhancing solution and anti-PrP antibody) and Benchmark LT automated system.

For blood collection (IACUC approved), the animals were restrained and a needle was inserted into the jugular vein. Immediately following blood collection (using sodium citrate as the anticoagulant), one half of the blood was chilled and shipped immediately. The remaining half of the collected whole blood sample was centrifuged at low speed for 15 min at 4° C. Plasma was removed, frozen and shipped on dry ice.

White-tailed deer care and sampling protocols were approved by the Colorado Division of Wildlife's (CDOW) IACUC. Neonatal white-tailed deer fawns acquired from several free-ranging sources were bottle-raised using canned evaporated bovine milk and established protocols (Wild and Miller 1991; Wild et al. 1994). Deer were confined to biosecure paddocks throughout the study, except during times of sample collections. Food, water and supplements were provided ad libitum in all paddocks. At about 6 months of age, white-tailed deer fawns were orally inoculated with about 0.5 g of conspecific, pooled, infectious brain material placed at the base of the tongue; previous analyses showed that this inoculum pool was infectious and contained about 6 μg PrP^{CWD} per g of brain tissue (Raymond et al. 2000; Wolfe et al. 2007). All deer were evaluated by a veterinarian experienced in recognizing clinical signs of CWD, and subjectively scored for behavioral changes, loss of body condition, ataxia, and salivation or polydipsia. The five deer for this study were heterozygous for glycine and serine at codon 96 of the native prion protein gene, had PrP^{CWD} accumulation in tonsil biopsies by 253 or 343 days post infection (dpi) (Wolfe et al. 2007), and were confirmed to be prion infected at postmortem examination 891 to 1774 dpi.

Blood samples were collected from the five inoculated white-tailed deer at 891 dpi. At the time of sampling, one animal (BC04) was in end-stage clinical chronic wasting disease, two (N204 and W1004) were showing some loss of body condition, and the other two (I304, K304) were clinically normal. For blood sampling, deer were sedated with xylazine, skin overlying the jugular vein was aseptically prepared, and about blood was collected via jugular venipuncture into a plastic bag treated with sodium citrate. Bags of blood were cooled and shipped overnight for processing.

### 2. Generation of Monoclonal Antibodies

PK-treated PrP^{Sc}, which consists of the core protein containing amino acids (aa) 90-231 (PrP₉₀₋₂₃₁), was isolated from the brains of 263K infected hamsters using a procedure originally reported by Hilmert and Diringer (1984) and modified by Rubenstein et al. (1994). This material was solubilized using guanidine hydrochloride extraction and methanol precipitated as previously described (Kang et al., 2003) and used as the immunogen. PrP^{-/-} mice were immunized and their immune responses monitored by ELISA as previously described (Kascsak et al., 1987). One of the immunized mice was used to produce hybridomas. The mouse received a final immunization of antigen by the intravenous route in phosphate-buffered saline (“PBS”) 4 days before fusion. Spleen cells were fused to an SP2/0 myeloma cell line expressing reduced levels of cell surface PrP^{C} (Kim et al., 2003). The hybridomas were screened by ELISA as previously described (Kascsak et al., 1987) and the resulting cells were cloned three times by limiting dilution. Large scale Mab production was carried out using disposable bioreactor flasks (Integra Biosciences, Switzerland) and antibody was purified from media using protein G immunoaffinity chromatography (Pierce, Rockford, Ill.). Protein was determined by the micro BCA protein assay (Pierce) and isotyping was performed using the mouse Mab isotyping kit (Pierce). Each of the Mabs was biotinylated using the EZ-link biotinylation kit (Pierce).

Numerous Mabs were generated using the solubilized PrP^{Sc} as immunogen and the low PrP expressing SP2/0 myeloma cell line. Three of these Mabs, 08-1/5D6 (5D6), 08-1/11F12 (11F12) and 08-1/8E9 (8E9) were selected for this study and have been isotyped as IgG1, IgG2b and IgG2b respectively. Individually, all three Mabs react with both the normal and disease associated PrP isoforms.

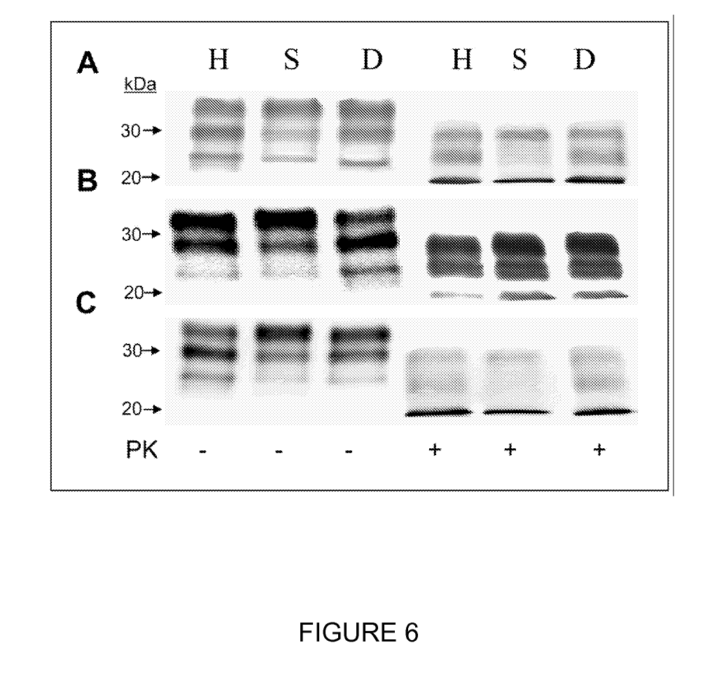

Western blotting of total brain lysates (FIG. 6) demonstrated that all three Mabs were reactive against PrP from non-protease treated brain samples and PK-treated PrP^{Sc} from 263K-infected hamsters, scrapie-infected sheep and CWD-infected deer (FIG. 6). Similar results were observed using untreated and PK-treated partially purified PrP^{Sc} preparations (data not shown). These Mabs were also immunoreactive against the normal and abnormal PrP isoforms and PK-treated PrP^{Sc} isolated from mouse brains infected with the ME7, 139A and 22L mouse-adapted scrapie strains and CJD-infected human brain as well as PrP^{C} derived from uninfected brain material from all the species tested including cattle (data not shown).

By indirect ELISA, the three Mabs were immunoreactive to PK-treated PrP^{Sc} purified from 263K-infected hamster brains. The degree of reactivity was dependent on the extent of the denaturation treatment. Either heat or SDS treatment alone increased immunoreactivity but a combination of the two treatments resulted in the highest levels of antibody binding and immunoreactivity (Table 1) approximating an additive effect of the two treatments and suggesting that epitope exposure is a multi-mechanistic process. Interestingly, although 5D6 binds to a conformational epitope, reactivity of this Mab is not lost, but rather enhanced upon PrP denaturation. It has previously been reported (Tayebi et al., 2004) that heat denaturation is not sufficient to disrupt the polymeric structure of PrP^{Sc}. Furthermore, the Mabs were equally immunoreactive by ELISA to both PrP^{C} from uninfected brains and total PrP (normal and abnormal PrP isoforms) in non-denatured brain homogenate. Immunoreactivity was equally enhanced approximately 2-fold following denaturation with SDS and heat. Following PK treatment and denaturation, the immunoreactivity of PrP^{Sc} was increased an additional 3-fold due to the presence of less exogenous brain protein binding as a result of the proteolytic digestion (data not shown).

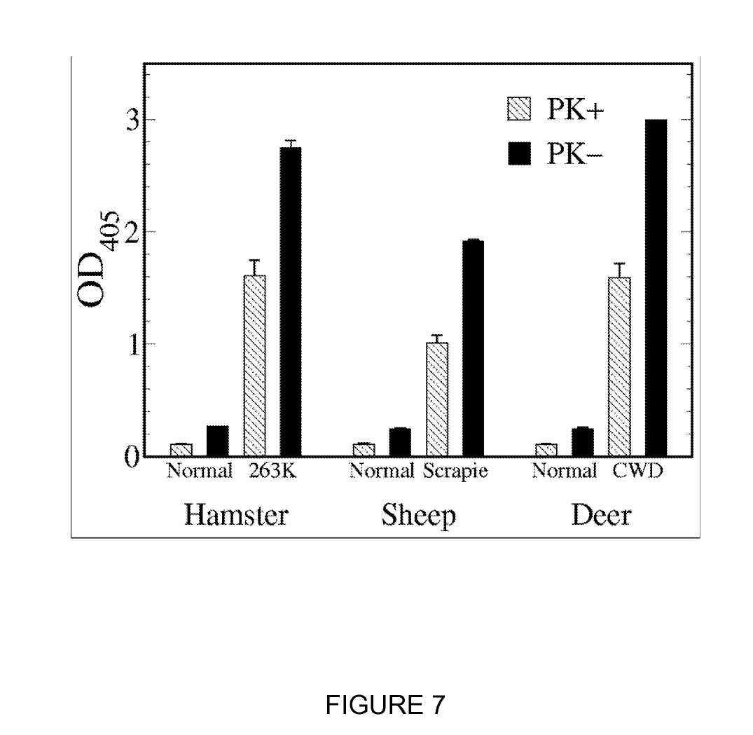

To increase specificity and sensitivity for PrP detection, a capture ELISA assay was used incorporating a biotinylated detection antibody. As expected, for each of the Mabs biotinylated, 5-6 biotins were bound to each antibody molecule. Further, the biotinylation of the Mabs did not interfere with or reduce their immunoreactivity as assessed by indirect ELISA using partially purified PK-treated PrP^{Sc} (data not shown). Therefore, any differences in the binding and reactivity of the detection antibodies are not the result of the physical biotinylation process. Using PK-treated PrP^{Sc} that had been denatured with SDS and heat, several Mab combinations were examined and each antibody was assessed both as the capture reagent and as the detection reagent (Table 2). Only one of the antibody combinations, Mab 11F12 as the capture reagent and biotinylated 5D6 as the detector, was successful in binding to and identifying PrP^{Sc}. The results were the same regardless of whether the PrP^{Sc} was derived from 263K-infected hamsters, scrapie sheep or CWD-affected deer. The capture ELISA assay utilizing the 11F12-5D6 Mab combination was next assessed for its ability to detect PrP in total and PK-treated brain homogenates from uninfected and infected hamsters, sheep and deer (FIG. 7). Similar to the results described above for the indirect ELISA assay on purified hamster brain PrP^{Sc}, the detection of PrP in the capture ELISA assay was also dependent on epitope availability and determined by the initial treatment of brain lysate. Untreated brain lysate from infected animals showed a slight (1.5-fold) increase in signal intensity compared to uninfected brain material whereas either detergent or heat denaturation alone resulted in a 4 to 7-fold increase. Not surprisingly, the highest levels (greater than 10-fold) of PrP^{Sc} detection were achieved when a combination of SDS and heat treatment were used. Furthermore, increasing the concentration of SDS above 1% reduced PrP^{Sc} detectability most likely due to an inhibition and/or reversal of antibody-antigen binding. This harsh denaturation treatment, as will be seen below, was not sufficient to completely destroy PrP conformation. It has previously been reported that scrapie infectivity, and presumably some degree of PrP^{Sc} conformation, could be maintained in purified PrP^{Sc} preparations following treatment with SDS, heat and SDS-PAGE (Brown et al., 1990; Rubenstein et al., 1994).

PrP^{C} could be detected in non-PK treated normal brain homogenates by capture ELISA from all three species. In all cases, the signal intensity (˜0.25-0.3) was no greater than twice above background (˜0.12-0.15). This material was eluted from the wells and examined by western blotting. In contrast to the results described above where PrP^{C} was detected directly from non-PK-treated brain homogenates, western blotting of eluted samples resulted only in the detection of IgG light and heavy chains. PrP^{C} was not detectable due to the low levels of bound material. Following PK digestion, ELISA values were reduced to background levels indicating the elimination of PrP^{C}. PrP^{Sc} could readily be detected by the capture ELISA assay in PK-treated brain homogenates from 263K-infected hamsters, sheep scrapie and CWD. Interestingly, capture ELISA assays performed on non-PK treated brain homogenates, which contain both PrP^{C} and PrP^{Sc}, showed signal intensities higher than what could be attributed to the PrP^{C} (determined from the non-PK normal tissue) and PrP^{Sc} (determined from the PK-treated infected tissue) aggregate (FIG. 7). It is possible that the increased signal intensity is due to the presence and binding of sPrP^{Sc}. An alternative explanation is that the binding of the protein, presumably full-length PrP^{Sc}, to the capture Mab induces a spatial change in the antigen which results in the epitope for the second Mab becoming more accessible. This process is referred to as positive immunocooperativity.

With the given set of Mabs used in this study, the degree of positive immunocooperativity, as shown in FIG. 7, was species dependent. PrP^{Sc} from CWD-infected deer showed the greatest levels with a 58% increase in 5D6 binding beyond that calculated solely from the combination of PrP^{C} and PrP^{Sc}, while sheep scrapie PrP^{Sc} showed a 46% increase. PrP^{Sc} from 263Kinfected hamsters exhibited the least, but still significant, with 40%. The values in FIG. 7 are based on triplicate readings for six individual samples for each species and expressed as the mean±standard deviation.

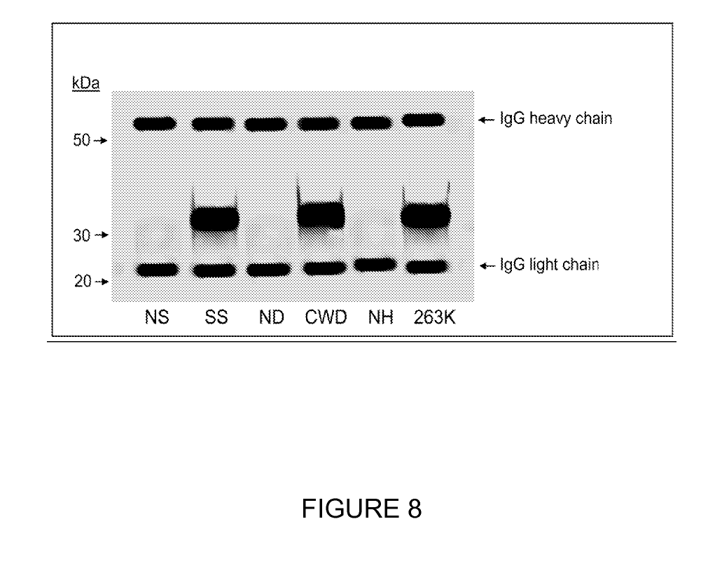

An antibody-induced spatial rearrangement and/or conformational change in PrP^{Sc} can be demonstrated by showing that the 11F12-5D6 captured material has altered the epitope for another PrP-specific Mab. The capture assay was performed on non-PK-treated, SDS and heat denatured PrP^{Sc}. This was followed by incubation with biotinylated Mab 8E9, streptavidinalkaline phosphatase and substrate. The lack of a signal above background indicated that the epitope for Mab 8E9 was either no longer available or accessible. However, elution of the 11F12-5D6 captured material from the microtiter wells followed by Western blotting and immunostaining with Mab 8E9 demonstrated robust PrP^{Sc} staining indicating that the Mab 8E9 epitope was once again available (FIG. 8). Presumably treatment with SDS-PAGE sample buffer, along with electrophoresis in the presence of SDS, alters the 11F12-5D6 binding to PrP^{Sc} and reverses the antibody-induced PrP^{Sc} changes to once again enable 8E9 binding.

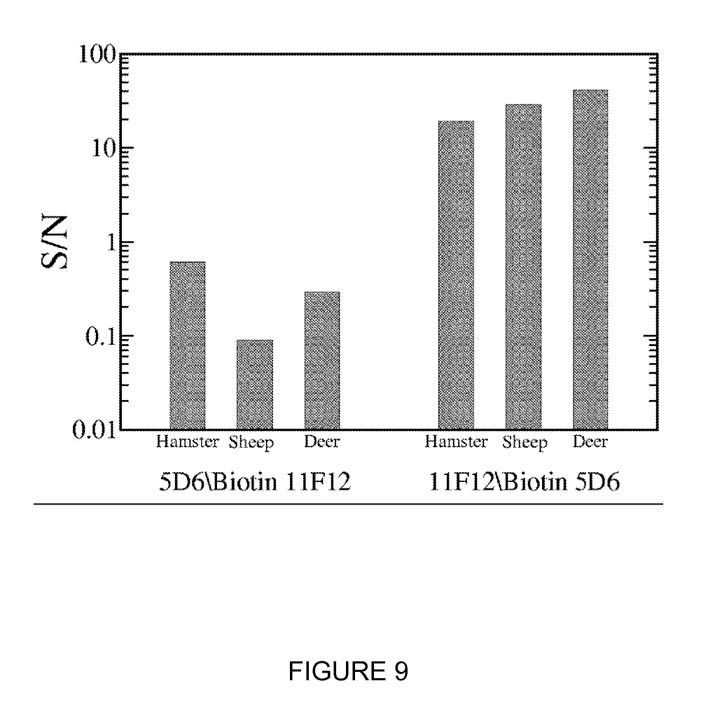

Although Mab 8E9 was able to bind to PrP^{C} and PrP^{Sc} directly on Western blots and indirect ELISA assays, replacing 5D6 with 8E9 in the capture ELISA assay resulted in no detectable PrP indicating the absence of biotinylated 8E9 binding to the antigen. Furthermore, the PrP^{Sc} specificity of the 11F12-5D6 antibody pair was not only due to the presence of these specific Mabs but also to the sequence of the binding events. Reversing the antibodies by utilizing 5D6 as the capture reagent and biotinylated 11F12 as the detection reagent (5D6/Biotin 11F12) resulted in minimal PrP^{Sc} binding from non-PK treated brain lysates when compared to the 11F12-biotinylated 5D6 combination (11F12/Biotin 5D6) (FIG. 9). A signal to noise (S/N) ratio was obtained by comparing the PrP signal obtained with the capture assay using infected brain lysates with the variance in the background signal obtained from uninfected material from hamster, sheep and deer brain tissue (S/N=(S−S0)/(3σS0); where S=signal, S0=mean background signal, σS0=standard deviation of the background signal). A S/N ratio of less than 1 indicates that a binding of the Mab is sufficiently weak that the signal measured contains a significant amount of noise. On the other hand, a S/N of 1 or greater indicates that the noise in the measurement is not significant indicating that most of the power in the measurement results from specific Mab binding. The confidence level increases exponentially as the S/N ratio increases. For the 5D6/Biotin 11F12 pair, the S/N ratios were approximately 0.6, 0.1 and 0.3 for hamster, sheep and deer, respectively, indicating that the Mab binding was nonspecific. However, with the 11F12/Biotin 5D6 combination the S/N ratios were approximately 19 (hamster), 28 (sheep) and 42 (deer). These ratios are indicative of the highly significant nature of the specific Mab binding. The values in FIG. 9 are based on triplicate readings for six individual samples for each species and the ELISA results calculated as the mean±standard deviation. The increased antibody binding from infected samples (based on the OD₄₀₅) are compared to the uninfected controls. Plotted on a logarithmic scale is the signal to noise ratio (S/N) as calculated from the signal power of the infected samples to the power in the control samples (noise).

### 3. Immunoassays

For the preparation of 10% brain homogenates, brain tissues were homogenized in 10 vol. of ice-cold lysis buffer (10 mM Tris-HCl, 150 mM NaCl, 1% Igepal™ CA-630 (Nonidet P-40), 0.5% deoxycholate, 5 mM EDTA, pH 8.0) in the presence of 1 mM phenylmethylsulfonyl fluoride (PMSF) (if the homogenate was to be treated with proteinase K (PK), PMSF was omitted from the lysis buffer). After centrifugation at 1,000×g for 10 min, the supernatants were aliquoted and stored at −80° C.

The protocol and reagents for the capture assays are described in Chang, B. et al., “PrP Antibody Binding-Induced Epitope Modulation Evokes Immunocooperativity,” J. Neuroimmunol. v. 205, issue 1-2, pp. 94-100 (2008), the contents of which are hereby incorporated herein in its entirety. Hybridoma cell lines producing the murine monoclonal antibodies used herein have been deposited as indicated, infra. For the capture ELISA assay, 96-well plates were coated with affinity-purified 11F12 capture monoclonal antibody (Mab) (5 μg/ml) at room temperature for 2-3 hrs. The coated wells were blocked with 3% bovine serum albumin (Sigma) in PBS overnight at 4° C. The wells were washed three times with PBST. The antigen was either non-PK- or PK-(100 μg/ml PK at 50° C. for 30 min) treated brain lysates to which was added a final concentration of 1% PMSF. All samples were treated with 1% SDS (final concentration), heated at 100° C. for 10 min. and centrifuged at 16,000×g for 5 min. The supernatants were serially diluted 10-fold and 100 μl was added to each well. The plates were incubated at 37° C. for 1 hr. The wells were washed three times with PBST and 100 μl of the biotinylated 5D6 detector Mab (5 μg/ml) was added. After 60 min the wells were washed with PBST and 100 μl streptavidin conjugated to alkaline phosphatase (1:5,000) was added for 60 min at 37° C. PNPP (4-Nitrophenyl phosphate disodium salt hexahydrate) (Sigma) substrate solution was added to each well (100 μl) and after 60 min, product was measured with an ELISA reader (Bio-Tek, Vermont, N.Y.) at OD₄₀₅.

For laser analysis, incubation with the biotinylated Mab 5D6 was followed by the addition of streptavidin conjugated to Rhodamine Red (1:1000). Following a 60 min incubation at 37° C., the wells were washed with PBST and treated with 100 μl 1N NaOH for 10 min at 100° C. and then shaken at room temperature for 20 min. The material was placed into a 100 ul Microcap™ (Drummond Scientific, Broomall, Pa.) microcapillary tube which was then inserted into a specifically designed tube sample holder for laser excitation and emission detection. Dilutions are calculated relative to the original starting brain tissue. Each value (data point) represents the mean±standard deviation (SD) from multiple assays as described in the figure legends.

### 4. Western Blotting

Ten percent brain homogenates were prepared in lysis buffer as described above. The samples were centrifuged at low speed (2000×g for 10 min). Ten microliters of the supernatants were mixed with a final of 1× sample buffer, heated at 100° C. for 4 min and subjected to SDS-polyacrylamide gel electrophoresis (SDS-PAGE) using 12% acrylamide gels, transferred to nitrocellulose membranes and immunostained using either streptavidin-conjugated to alkaline phosphatase with NBT and BCIP as the substrate (Kascsak et al., 1986) or horseradish peroxidase-conjugated goat anti-mouse IgG (Pierce™) with super signal west femto maximum sensitivity substrate (Pierce) as previously described (LaFauci et al., 2006). For samples that were PK digested prior to SDS-PAGE, 40 ul of the supernatants from the low speed centrifugation were incubated with 100 μg/ml PK (final concentration) for 30 min at 50° C. followed by the addition of 1% PMSF, 1×SDS-PAGE sample buffer and heating at 100° C. for 5 min.

### 5. Immunoprecipitation

MagnaBind protein G beads (Pierce) were washed 3 times with PBS, resuspended in 50 μl of PBS and 200 μl of 10% brain homogenate was added with 50 μg of Mab 8E9 (10 mg/ml) in a total volume of 1.2 ml PBS. After mixing at room temperature for 1 hr, the beads were magnetically separated, washed 3 times with PBS containing 0.2% Tween 20 (PBST) and then resuspended in 600 μl PBS. After heating at 100° C. for 10 min and microcentrifugation at 16,000×g for 3 min, the supernatants were used for capture ELISA.

For blood samples, magnaBind protein G beads were resuspended in 100 μl PBS, followed by the addition of 100 μg Mab 8E9 in a final volume of 5 ml PBS and mixed at room temperature for 1 hr. The beads were washed with PBST, resuspended in 5 ml PBS containing 500 μl plasma and incubated for an additional 1 hr. As described above for brain, the beads were isolated, washed in PBST, heated and the microcentrifuged supernatant analyzed by capture ELISA.

### 6. Protein Misfolding Cyclic Amplification (PMCA)

As a source of PrP^{C} for sPMCA of both brain and blood, 10% (wt/vol) brain homogenates from normal hamsters, sheep and deer [prepared in PBS containing 150 mM NaCl, 1.0% Triton X-100, 4 mM EDTA, and complete protease inhibitor cocktail (Calbiochem)] were centrifuged (1,500×g, 30 sec) and the supernatants quick frozen. A 500 μl aliquot of serial 10-fold dilutions (10-8 to 10-11) of brain homogenates from 263K-infected hamster, sheep scrapie and CWD deer were mixed with 100 μl of the 10% normal brain supernatant from the corresponding species and incubated (1 hr at 37° C.) with shaking Each sample was then sonicated (28 W power output) followed by the addition of another 100 μl of 10% normal brain homogenate and incubation (1 hr at 37° C. with shaking) This was defined as one cycle of amplification. After each round of 5 cycles, PMCA was continued by transferring 500 μl of the PMCA reaction mix from the original reaction tube to new tube and adding 100 μl 10% normal brain homogenate. PMCA on 500 μl aliquots of undiluted scrapie sheep or CWD deer plasma was carried out similarly as described for brain. Following PMCA, samples were centrifuged at 2,000×g for 10 min. For brain samples, 200 μl of supernatant was digested with proteinase K (PK) (100 μg/ml, 500 C, 30 min), followed by the addition of 1% protease inhibitor cocktail and 1% SDS. Samples were heated at 100° C. for 10 min and 10 μl aliquots were analyzed by western blotting (Chang et al. 2009). For blood, following PMCA 500 μl of amplified blood samples were either untreated or PK-treated followed by Mab 8E9 immunoprecipitation prior to analysis by western blotting or SOFIA. For all PMCA samples, dilutions were expressed relative to the original undiluted brain or blood samples. Blood samples not subjected to PMCA were immunoprecipitated and processed similarly as above without the sonication and 37° C. incubation cycling.

### 7. SOFIA

Ninety-six well High Binding plates (Costar, N.Y.) were coated with capture Mab 11F12 (5 μg/well in 100 μl) at room temperature for 3 hrs and blocked with casein in TBS overnight at 4° C. Magnetic protein G beads and Mab 8E9 were mixed for 60 min, washed 3 times with PBST and the pellets resuspended in 800 μl PBS. Blood samples were centrifuged (800×g, 5 min) and 800 μl of the supernatants were combined with SDS (1% final conc), heated at 100° C. for 10 min, mixed with the G beads-Mab 8E9 for 60 min and wash 3 times with PBST. The final washed pellets were resuspended in 800 μl PBS and heated for 10 min. After centrifugation at 16,000×g for 5 min, 100 μl of supernatant was added to each well. After incubation at room temperature (1 hr) and washing with PBST, 100 μl of biotinylated Mab 5D6 (2 μg/well) was added for 1 hr. followed by washing and incubation with 100 μl streptavidin-Rhodamine Red-X conjugate (Invitrogen) for 1 hr. After 4 PBST washes, 100 μl of 1N NaOH was added and the plates were heated (100° C. for 10 min), mixed for 30 min at room temperature and neutralized with equimolar amounts of HCl. Analysis was performed on 90 μl aliquots.

### 8. Instrumentation

The setup is designed around a commonly used disposable 100 microliter micro-capillary (Drummond Scientific Co., Broomall, Pa.) as a sample holder. The sample is excited by focusing temporally modulated light from a solid state, frequency-doubled Nd:YAG laser (Beam of Light Tech.™, Clackamas, Oreg.) along the axis of the capillary, with typical power of 30 mW continuous wave at a wavelength of 532 nm, which matches well with the absorption peak of Rhodamine. A fiber optic assembly was designed comprised of four linear arrays which span approximately a third of the length of the capillary and are positioned at 90 degrees with respect to each other around the perimeter of the capillary. Because of the large numerical aperture (0.22, or an acceptance angle of ˜23 deg.) of the fibers, this orientation of the fibers results in complete coverage of the sample's field of view. The light collected by the four linear arrays is ganged (i.e., bundled, or combined) and focused into transfer optics in which a holographic notch filter (Kaiser Optical Systems Inc. Ann Arbor, Mich.), and band pass filters (Omega Optical, Inc. Brattleboro, Vt.) are mounted. These are used to eliminate the scattered light from the excitation source, and band-limit the detection of the fluorescence of the reporter dye, respectively. The light is then focused back into a single, multi-mode, 400 micron optical fiber (Thorlabs™, Inc. Newton, N.J.) and coupled to a single low noise photo-voltaic diode detector (United Detector Technology, Hawthorne, Calif.) which is mounted on a BNC connector directly on the pre-amplifier of the detection electronics. Detection of the signal employs a phase sensitive, or “lock-in”, detection scheme. The excitation source is modulated with an optical chopper (Thorlabs Inc.) which serves to generate the reference frequency for the detection system. The diode detector is mounted on the input of the transconductance pre-amplifier (Stanford Research Systems, Inc. Sunnyvale, Calif.) to reduce the total line impedance and eliminate difficulties in impedance matching of the signal at these low levels. The signal is then detected with a lock-in amplifier (Stanford Research Systems) and data acquisition is performed through a LabView™ (National Instruments Inc., Austin, Tex.). The program consists of an electronic strip chart which poles the lock-in amplifier for its reading in voltage periodically displays the time history of the measurements to the operator, and stores the values with a time stamp in an ASCII file. The time constant of the lock-in amplifier should be chosen to provide a bandwidth of a few tenths of a Hertz. For these measurements a time constant of 3 seconds was chosen. The lock-in requires several time constants in duration to obtain a stable reading (3 to 30 seconds in this case). The values for the measurements were taken after the signal had stabilized (20 to 30 sec.) after loading a new sample. The modulation of the excitation source, and reference frequency for the lock-in detector, were 753 Hz which was chosen to minimize environmental noise. In addition to this filtering of the signal at line-frequency and two times line frequency was done with the lock-in amplifier and the pre-amplifier signal was band-pass filtered at the modulation frequency. For the samples the pre-amplifier sensitivity of 1 nA/V was chosen, giving an input impedance of 1 M Ohm. In making the measurements a set of startup procedures was maintained which included: a warm up of 15 minutes for all electronics (the laser, lock-in amplifier, pre-amplifier), a visual check of dark signal levels to assure that system is properly electrically grounded, a measurement of laser power to check for stability and output level, a visual check of laser alignment. Control measurement of baseline signal is checked using a capillary with distilled, deionized water.

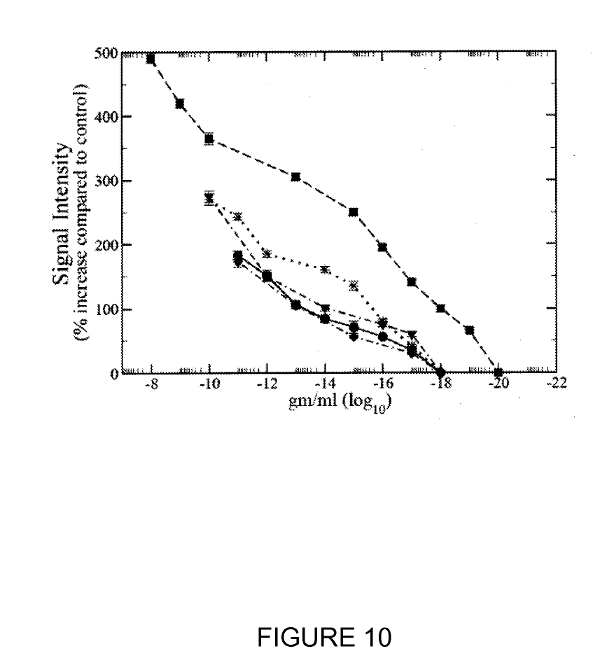

The sensitivity limits of the instrument were tested by measuring the fluorescence signal emission of Rhodamine Red at decreasing concentrations. Rhodamine Red was detectable to a concentration of 0.01 attograms (ag) [20 attomoles (am)] (FIG. 10). Determination of specificity and sensitivity was carried out by performing assays using full-length recombinant PrP (rPrP) from deer, hamster, mouse and sheep. Regardless of the species tested, the limits of detectability were ≧10 ag rPrP. Turning to FIG. 10, data was obtained on the instrument of FIG. 1, wherein dilutions of Rhodamine Red (▪) in water were added to 100 μl micro-capillary tubes, and surround optical fiber fluorescent signal emission was recorded. The relative signal intensities were calculated based on the fluorescence signal emission of water alone. In the case of the rPrP from mouse (*), hamster (♦), sheep (▾) and deer (), the rPrP was diluted in 1% PrP^{-/-} brain homogenate and subjected to SOFIA. The relative fluorescent signal intensities were calculated based on similar assays performed with rPrP diluent (1% PrP^{-/-} brain homogenate) alone. Triplicate assays at a preamplifier setting of 1 nA/V were performed for each rPrP concentration and the data was plotted as the mean of the signal intensities (% increase compared to control) ±SD.

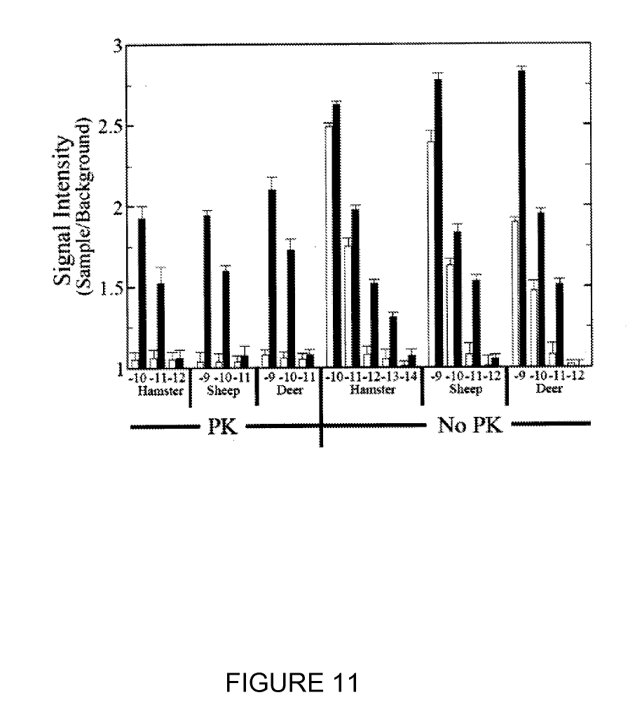

Brain homogenates from normal and infected hamsters, deer and sheep were examined for their use in the method of the present invention. Western blotting of 10% brain homogenates confirmed the presence of PrP^{Sc} in the starting material. Typical PrP banding patterns were evident in the 10% brain homogenates prior to PK treatment with the characteristic band shifting to lower molecular sizes of PrP^{Sc} following PK digestion along with the elimination of PrP^{C} from the normal hamster brain material as confirmation of complete proteolytic digestion. Serial dilutions of detergent extracted brain homogenates from clinical animals have demonstrated that the limits of PrP^{Sc} detection by Western blotting is approximately 10⁻³-10⁻⁴ while detection of PrP^{Sc} by capture ELISA was sensitive following an additional 10¹-10² fold dilution (data not shown). In comparison, using the same Mabs and brain homogenates, the sensitivity of the assay reported in this manuscript exceeded that for Western blotting and capture ELISA by at least 5 orders of magnitude. Using the method of the present invention, the signal to baseline ratios (S/B) were used to evaluate PrP detectability in brain homogenates. It was determined that an S/B ratio of greater than 1.1 indicated the presence of PrP. Serial dilutions of PK-treated and untreated brain homogenates from normal and infected brain tissue of hamsters, sheep and deer were assayed by the method of the present invention (FIG. 11). Values were expressed as a ratio of signal from the samples' Rhodamine Red fluorescence emission (S) vs. background baseline signal derived from fluorescent emission of the diluent (1% PrP^{-/-} brain homogenate or homogenizing buffer) alone (B). The data represents the mean±SD from three independent experiments, each performed in triplicate at a preamplifier setting of 1 nA/V, for each brain homogenate dilution. As expected, following PK treatment all samples from normal brain tissues had S/B ratios of less than 1.1 regardless of the concentration tested indicating the absence of PrP^{C}. As demonstrated by total signal output or S/B ratios above 1.1, protease resistant PrP^{Sc}, from serial 10-fold dilutions of PK-treated infected hamster brain homogenates, was detectable to a dilution of 10⁻¹¹ and from sheep and deer to 10⁻¹⁰. In addition, maximum PrP^{Sc} detection from the PK-treated brain homogenates ranged from dilutions of 10⁻⁷-10⁻⁸ for hamsters as well as sheep and deer.

In the case of 10-fold serially diluted non-PK treated normal brain homogenates, PrP^{C} was detectable by SOFIA to a dilution of 10⁻¹¹ for hamsters and 10⁻¹⁰ for deer and sheep (with peak detection at 10⁻⁶-10⁻⁷ dilutions) after which the S/B ratios all fell below 1.1. The S/B ratios from of non-PK treated brain tissue of 263K infected hamsters, scrapie-infected sheep and CWD-infected deer continued to indicate the presence of PrP. Serially diluted brain homogenates from infected tissues all showed S/B values greater than 1.1 to a dilution of 10⁻¹¹ for sheep and deer (with peak detection at 10⁻⁷) and 10⁻¹³ for hamsters (peak detection at 10⁻⁸). These results indicate that PrP from non-protease treated, infected brain tissue can be diluted beyond the levels of PrP^{C} detectability while still maintaining the capability to detect total PrP^{Sc}. These results further suggest that there is at least 1 log more total PrP^{Sc} than PrP^{C} in an infected brain at clinical disease. In support of this, it has previously been reported that PrP^{Sc} accumulates in the brain during scrapie infection and attains concentrations 10 times greater than that of PrP^{C}. Using previously published data on 263K-infected hamsters (R. Atarashi et al. “Ultrasensitive detection of scrapie prion protein using seeded conversion of recombinant prion protein,” Nature Meth. vol. 4 (2007) pp. 645-650), SOFIA has a detection limit of approximately 10 ag of PrP^{Sc} from non-PK treated hamster brain. Extrapolation directly from the hamster data suggests that 1 femtogram of PrP^{Sc} can be detected from sheep and deer brain material. However, assuming equal antibody reactivity, Western blotting of diluted samples indicated that there is at least 10-100 fold more PrP^{Sc} in hamster brains than in sheep and deer brain material on a gram equivalent basis (data not shown) suggesting that detection of the protein in the latter two species could be in the range of 10-100 ag or better.

### 9. Detection of PrP^{Sc} in Blood

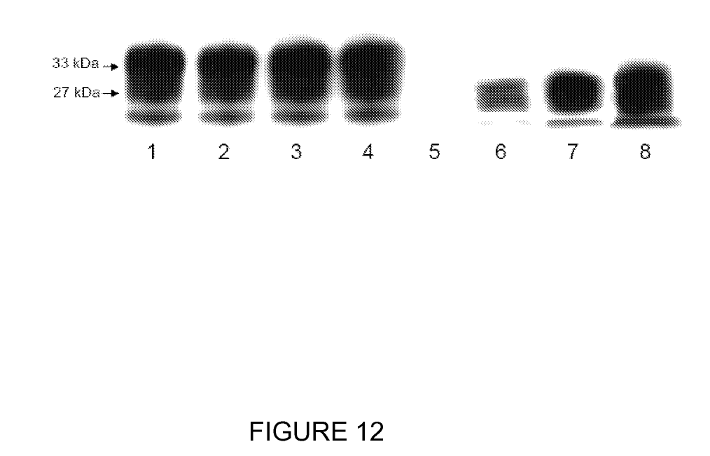

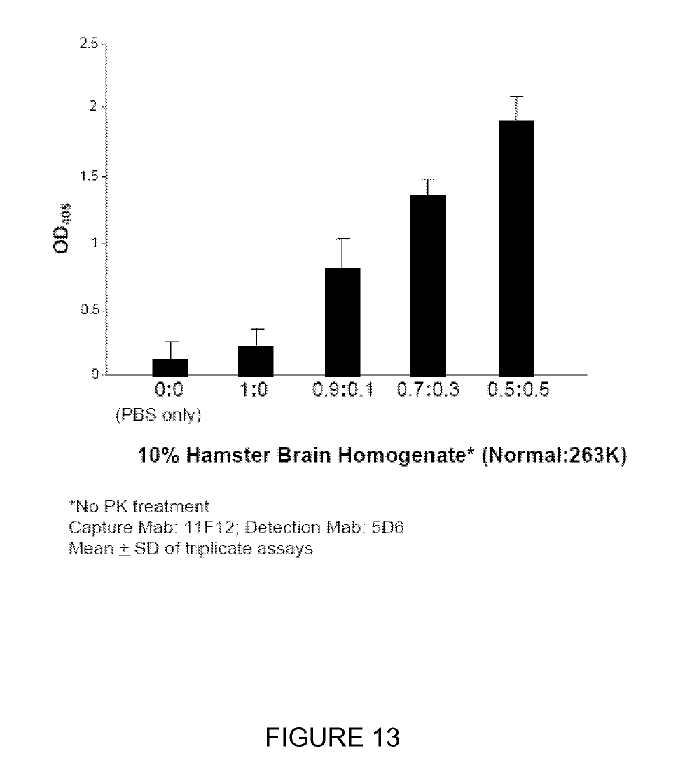

Immunoprecipitation with Mab 8E9 serves as a bridge linking PMCA and SOFIA, so, the utility of this Mab for PrP immunoprecipitations was examined. Mixtures of various ratios of 10% brain homogenates from uninfected and 263K-infected hamsters (uninfected:infected (%)-100:0, 90:10, 70:30, 50:50) were immunoprecipitated and analyzed by western blotting (FIG. 12). Ten percent normal brain homogenates (NBH) and 263K-infected hamster brain homogenates (263K BH) were combined in various proportions (lanes 1, 5-NBH only; lanes 2, 6-90 μL, NBH and 10 μL, 263K BH; lanes 3, 7-70 μL, NBH+30 μL263 BH; lanes 4, 8-50 μL, NBH+50 μL, 263K BH) and immunoprecipitated with Mab 8E9. The immunoprecipitated samples were either untreated (lanes 1-4) or PK-treated (lanes 5-8) prior to western blotting and immunostaining with Mab 11F12. In the absence of PK digestion, all samples, regardless of the brain homogenate ratios, showed similar immunostaining intensities (lanes 1-4). Western blots of PK-treated immunoprecipitants (lanes 5-8) demonstrated similar immunostaining when directly compared to samples containing only PK-treated 263K-infected brain homogenates (data not shown). These results indicate that Mab 8E9 immunoprecipitated both PrP^{C} and PrP^{Sc} and the presence of PrP^{C} did not inhibit or reduce maximal PrP^{Sc} immunoprecipitation. To quantitatively and qualitatively evaluate the PrP isolated, capture ELISA was performed on Mab 8E9 immunoprecipitants from the combinations of PK-untreated normal and 263K-infected hamster brain homogenates (FIG. 13). The capture ELISA utilized the same Mab pair (11F12 as the capture Mab and 5D6 as the detector Mab) as that used for SOFIA. Mab 8E9 immunoprecipitation of the normal brain:infected brain combinations followed by capture ELISA resulted in increasing ELISA signal intensities as the levels of infected brain material increased in the starting mixtures. Since the brain material was not proteolytically digested, each mixture contained either PrP^{C} alone or a mixture of both PrP^{C} and PrP^{Sc}, as confirmed by western blotting (FIG. 12). However, analysis of the immunoprecipitants by the capture ELISA indicates that the increasing signal intensities are dependent on the presence of PrP^{Sc} and not PrP^{C}. This points to the utility of these specific Mabs and the methodology for the detection of PrP^{Sc}. Although the immunoprecipitation-capture ELISA format could readily detect brain-derived PrP^{Sc} from 263K-infected hamsters, scrapie sheep and CWD deer, PrP^{Sc} could not be detected in blood from clinical animals.

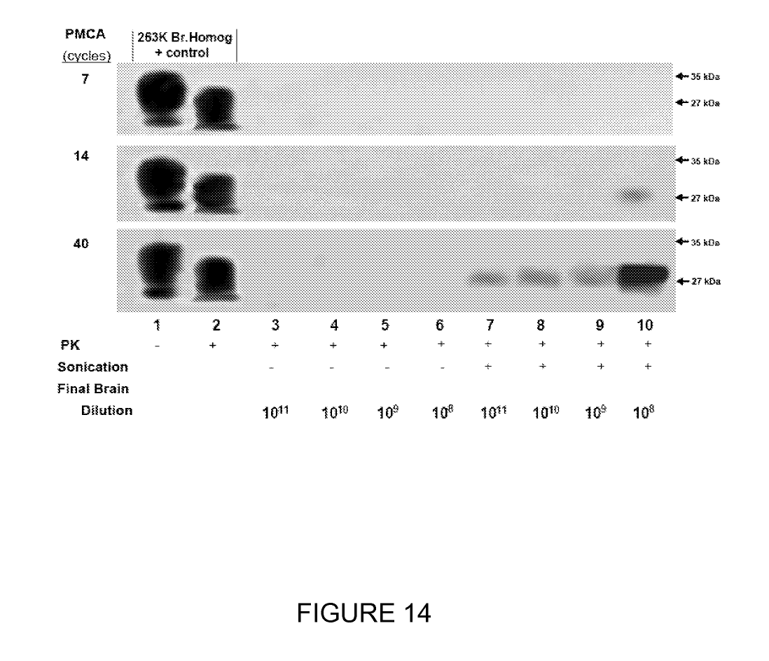

The ability to detect PrP^{Sc} in blood from 263K-infected hamsters and sheep scrapie samples following serial PMCA (sPMCA) has previously been reported. However, the large number of PMCA cycles necessary for PrP^{Sc} detection makes the technique impractical for use as a diagnostic assay. The issue of PrP^{Sc} detection in blood has been approached by incorporating sPMCA, followed by immunoprecipitation of the amplified target, and detection with the sensitive SOFIA assay (Chang et al., 2009). sPMCA was evaluated and validated using hamster brain (FIG. 14)). Dilutions of hamster brain homogenates were subjected to 7, 14 and 40 cycles of PMCA (lanes 7-10) while identical samples were processed similarly without sonication (lanes 3-6). Ten μL aliquots of each sample was analyzed by western blotting with Mab 11F12. Control samples were 10⁻² dilutions of 263K-infected hamster brain homogenates without (lane 1) and with (lane 2) PK digestion prior to western blotting 10⁻⁸-10⁻¹¹ dilutions (relative to the original brain tissue) of 10% 263K-infected hamster brain homogenate were subjected to sPMCA using normal hamster brain homogenate as a PrP^{C} source. PrP^{Sc} was undetectable at all the dilutions following 7 cycles of sPMCA but could be detected in the 10⁻⁸ diluted sample by the completion of 14 cycles (FIG. 14, lane 10). After 40 cycles of sPMCA (sPMCA₄₀), PK-resistant PrP^{Sc} was detectable at all dilutions of 263K-infected brain homogenates tested (FIG. 14 lanes 7-10). Similarly diluted hamster brain homogenates that were processed in parallel with the PMCA sonication steps omitted, did not show any PrP^{Sc} amplification as demonstrated by the absence of PK-resistant PrP^{Sc} immunostaining (FIG. 3, lanes 3-6). Comparing detection limits of PrP^{Sc} in the absence of PMCA (10⁻⁶ relative to the original brain tissue) with detection after sPMCA₄₀, and taking into account the sample size analyzed, the results estimate a 10⁴ fold amplification as a result of PMCA.

Similar PMCA experiments with diluted sheep brain or CWD brain homogenates from clinical animals (along with the uninfected brain homogenates of the corresponding species as the source of PrP^{C}) demonstrated the initial detection of amplified PrP^{Sc} by western blotting at 28 cycles of PMCA at the 10⁻⁸ dilution of infected brain. The increased number of cycles needed for the initial detection of PrP^{Sc} from sheep and deer brain compared to hamster brain was due to the lesser amount of starting PrP^{Sc} found in the original brain tissue. As expected, by the end of sPMCA₄₀, the amplified PrP^{Sc} from the scrapie sheep and CWD deer brain homogenates was demonstrated by increased immunostaining intensity and detection limits of PK-resistant PrP^{Sc}. Although the sheep and deer brain tissue contained less PrP^{Sc} compared to infected hamster brain, the levels of amplification were still approximately 4 logs relative to the initial PrP^{Sc} levels (not shown).

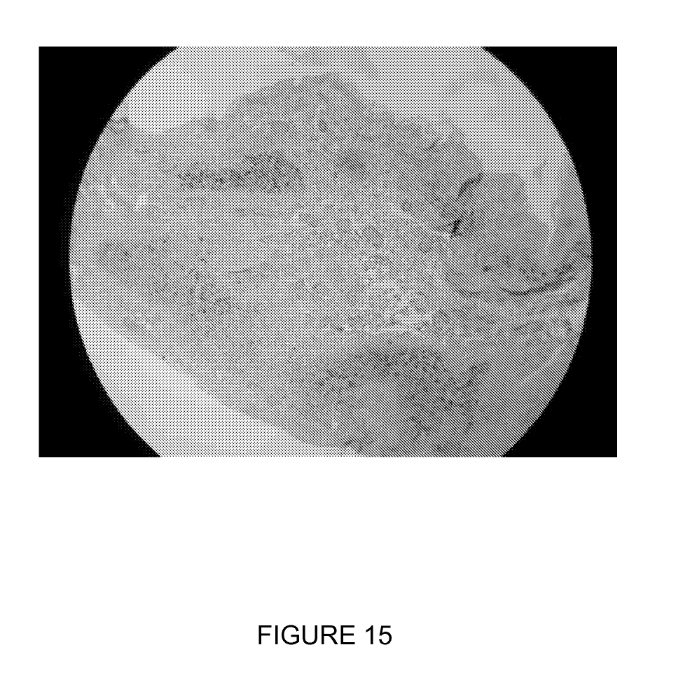

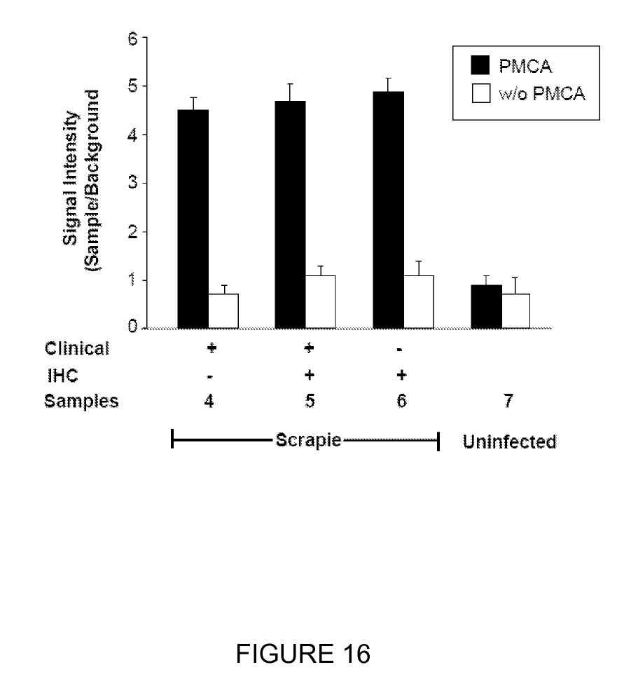

Plasma from scrapie sheep and CWD deer were subjected to sPMCA₄₀. The sheep samples consisted of three groups (Table 3, groups 1-3) of scrapie sheep, which, at the time of blood collection, were differentiated based on the presence or absence of clinical signs and PrP^{Sc} immunohistochemical (IHC) staining of third eyelid lymphoid follicles (FIG. 15). All animals in group 3 that did not display clinical symptoms at the blood collection time points eventually progressed to clinical disease. The group of uninfected sheep (Table 3, group 4) were housed and maintained in an isolated, scrapie-free area. CWD samples consisted of several experimentally infected (oral route) preclinical and clinical white-tailed deer (Table 4). All of the sheep and CWD samples were individually subjected to sPMCA₄₀ and analyzed by western blotting following PK digestion. Following sPMCA₄₀ of plasma, western blotting of PK-treated PMCA products either prior to or after PrP^{Sc} concentration by Mab 8E9 immunoprecipitation, did not reveal any PrP^{Sc}. The addition of polyadenylic acid [poly(A)], which has been reported to facilitate rapid detection of low levels of PrP^{Sc} from sheep blood (Thorne and Terry, 2008), did not improve amplification efficiency to the point of PrP^{Sc} detection from sheep scrapie or CWD deer plasma following sPMCA₄₀. The lack of PrP^{Sc} detection following sPMCA₄₀ from sheep blood was independent of the sheep genotypes used (data not shown). That is, the pairing of sheep genotypes between the source of PrP^{Sc} and the normal sheep brain PrP^{C} did not sufficiently increase the amplified product for detection by immunoblotting. Since western blotting was not informative, it is unclear whether PrP^{Sc} was initially present in the blood from CWD and any of the animals comprising the three groups of sheep scrapie or whether PMCA was successful but western blotting was not sensitive enough to detect the amplified PrP^{Sc} after only 40 cycles. It has been reported that PMCA of sheep blood could lead to false positive results due to the apparent spontaneous generation of PrP^{Sc} (Thorne and Terry, 2008). Therefore, rather than continue increasing the number of PMCA cycles, surround optical fiber immunoassay (SOFIA) (Chang et al., 2009) was used for PrPSc detection of untreated and PK-treated Mab 8E9 immunoprecipitated sPMCA₄₀ products. Our studies demonstrated that in the absence of sPMCA₄₀, the readings obtained by SOFIA from scrapie sheep and uninfected sheep plasma samples were similar and approached baseline levels (FIG. 16). Prior to sPMCA₄₀, the SOFIA signal intensities (sample/background) for the individual samples ranged from 0.5-0.9 (group 1), 0.7-1.2 (group 2), 0.8-1.3 (group 3) and 0.6-1.1 (group 4). Since previous studies on the dynamic range of SOFIA (Chang et al., 2009) demonstrated that in PK-untreated clinical sheep brain PrP^{Sc} was detectable in femtomole range (Chang et al., 2009), it is likely that the levels of PrP^{Sc} in scrapie sheep plasma samples are below the detectable range. In an attempt to amplify the levels of PrP^{Sc} to within the dynamic range of SOFIA, sPMCA40 was performed followed by Mab 8E9 immunoprecipitation. Non PK-treated PrP^{Sc} could be detected by SOFIA on the immunoprecipitated sPMCA₄₀ products (FIG. 16). Following sPMCA₄₀, the range of signal intensities (0.7-1.2) for the individual samples of the control group (group 4) did not significantly differ from those samples prior to PMCA. However, the range of SOFIA signal intensities for all three groups of scrapie sheep were similar to each other (group 1: 4.3-4.8, group 2: 4.4-5.1), group 3: 4.8-5.3), regardless of their clinical manifestations, and significantly greater than both the pre-PMCA values as well as the uninfected samples (group 4). The value of this approach is realized when one considers that confirmation of disease was dependent on the sheep being scrapie infected but was independent of the presence of clinical signs and the neuropathology as all three groups of sheep tested positive for the presence of PrP^{Sc} (FIG. 16). PrP^{Sc} amplification was also independent of genotype compatibility since there was no difference in the amplification when normal brain homogenates from either ARQ/ARQ or ARQ/VRQ sheep were used with any of the infected sheep plasma samples. Furthermore, the need for PK digestion to distinguish PrP^{C} from PrP^{Sc} was unnecessary since the results of SOFIA were the same regardless of whether the sPMCA₄₀ products were untreated (FIG. 16) or PK-treated (not shown) prior to immunoprecipitation and immunoassay analysis. The data in FIG. 16 was generated by dividing plasma samples into 3 groups according to the appearance of clinical signs and immunohistochemistry (IHC) associated with sheep scrapie. Each plasma sample was subjected to PMCA₄₀ (▪) or incubated without PMCA (□). Each sample was either untreated or PK digested followed by Mab 8E9 immunoprecipitation and analysis of PrPSc by SOFIA. Plasma samples from each of the 3 groups was assayed in triplicate and the data for all the samples in each group combined and expressed as mean±standard deviation.

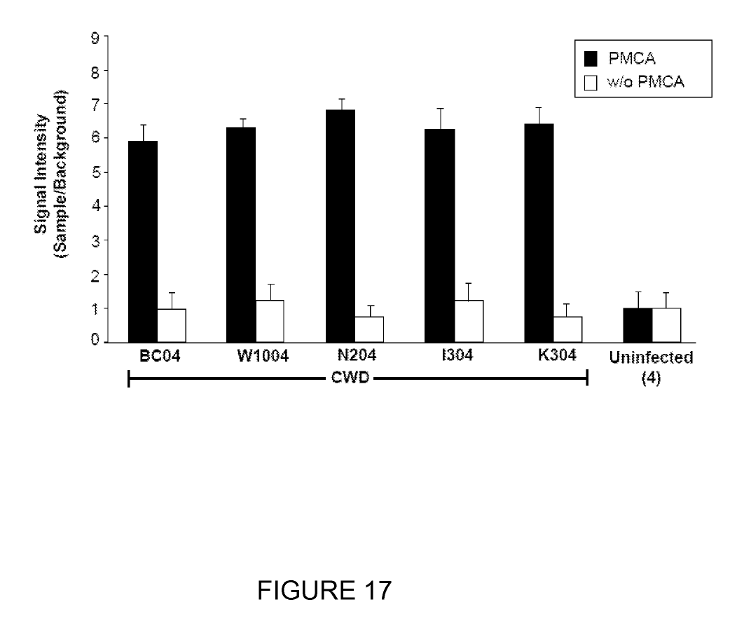

Similar studies were carried out with plasma obtained from several preclinical and clinical cases of deer CWD (FIG. 17). Similar to the sheep plasma samples described above, the signals obtained by SOFIA on the CWD samples in the absence of sPMCA₄₀ did not differ from the uninfected controls, which themselves approached background. In addition, PK-resistant PrP^{Sc} could not be detected by either capture ELISA or western blotting following sPMCA₄₀. However following immunoprecipitation of the sPMCA₄₀ products, PrP^{Sc} was detectable by SOFIA from all preclinical and clinical CWD blood (FIG. 17). Furthermore, similar to the scrapie sheep samples, the SOFIA values were dependent on the samples originating from infected animals but confirmation of disease by SOFIA was independent of the clinical status of the diseased animal. The data in FIG. 17 was generated by subjecting each of the plasma samples from the five CWD cases to sPMCA₄₀ (▪) or maintained in the absence of PMCA (□). All samples were either undigested or PK treated followed by Mab 8E9 immunoprecipitation and SOFIA. Results are shown for the PK-untreated samples and the values represent the mean of triplicate assays ±SD. In the case of the 4 uninfected deer plasma samples, each of the 4 samples was analyzed in triplicate and the combined results of the 4 samples are expressed as the mean±SD. In all embodiments of the present invention, all percentages are by weight of the total composition, unless specifically stated otherwise.

All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### REFERENCE LIST

Atarashi, R. et al. “Simplified ultrasensitive prion detection by recombinant PrP conversion with shaking,” Nat. Methods 5, 211-212 (2008).
Bieschke, J. et al., “Autocatalytic self-propagation of misfolded prion protein,” Proc. Natl. Acad. Sci. U.S.A. 101, 12207-12211 (2004).
Brown, P. et al. “Conservation of infectivity in purified fibrillary extracts of scrapie-infected hamster brain after sequential enzymatic digestion or polyacrylamide gel electrophoresis,” Proc. Natl. Acad. Sci. U.S.A. 87, 7240-7244 (1990)
Brown, D. R. et al., “Normal prion protein has an activity like that of superoxide dismutase,” Biochem. J. 344, 1-5 (1999). Erratum in: Biochem. J. 345, 767 (2000).
Bueler, H. et al., “High prion and PrPSc levels but delayed onset of disease in scrapie-inoculated mice heterozygous for a disrupted PrP gene,” Mol. Medicine 1, 19-30 (1994).
Bulgin, M. S. et al., “Association between incubation time and genotype in sheep experimentally inoculated with scrapie-positive brain homogenate,” Am. J. Vet. Res. 67, 498-504 (2006)
Carlson, G. A. et al., “Prion isolate specified allotypic interactions between the cellular and scrapie prion proteins in congenic and transgenic mice,” Proc. Natl. Acad. Sci. U.S.A. 91, 5690-5694 (1994).
Castilla, J. et al. “In vitro generation of infectious scrapie prions,” Cell 121, 195-206 (2005a).
Castilla, J. et al. “Detection of prions in blood,” Nat. Medicine. 11, 982-985 (2005b).
Chang, B. et al. “Surround optical fiber immunoassay (SOFIA): more than an ultra-sensitive assay for PrP detection,” J. Virol. Methods 159, 15-22 (2009).
Chang, B. et al. “PrP antibody binding-induced epitope modulation evokes immunocooperativity,” J. Neuroimmunol. 205, 94-100 (2008).
Deleault, N. R. et al., “Protease-resistant prion protein amplification reconstituted with partially purified substrates and synthetic polyanions,” J. Biol. Chem. 280, 26873-26879 (2003).
Gavier-Widen, D. et al., “Diagnosis of transmissible spongiform encephalopathies in animals: a review,” J. Vet. Diagn. Invest. 17, 509-527 (2005).
Haley, N. J. et al. “Detection of sub-clinical CWD infection in conventional test-negative deer long after oral exposure to urine and feces from CWD+deer,” PLoS One 4, e7990 (2009).
Haley, N. J. et al., “Detection of CWD prions in urine and saliva of deer by transgenic mouse bioassay,” PLoS One 4, e4848 (2009).
Hilmert, H. and Diringer, H. “A rapid and efficient method to enrich SAF-protein from scrapie brains of hamster,” Biosci. Rep. 2, 165-70 (1984).
Houston, F. et al. “Transmission of BSE by blood transfusion in sheep,” Lancet 356, 999-1000 (2000).
Jones, C. E. et al. “Preferential Cu2+ coordination by His96 and His111 induces beta-sheet formation in the sunstructured amyloidogenic region of prion protein,” J. Biol. Chem. 279, 32018-27 (2004).
Jones, M. et al. “In vitro amplification and detection of variant Creutzfeldt-Jakob disease PrP(Sc),” J. Pathol. 213, 21-26 (2007).
Kang, S. C. et al. :Guanidine hydrocholide extraction and detection of prion proteins in mouse and hamster prion diseases by ELISA,” J. Pathol. 199, 534-41 (2003).
Kascsak, R. J. et al. “Immunological comparison of scrapie-associated fibrils isolated from animals infected with four different scrapie strains”. J. Virol. 59, 676-683 (1986).
Kascsak, R. J. et al. “Mouse polyclonal and monoclonal antibody to scrapie-associated fibril proteins,” J. Virol. 61, 3688-3693 (1987).
Kim, J. I. et al. “Comparison of PrP transcription and translation in two murine myeloma cell lines,” J. Neuroimmunol. 140:137-142 (2003).
Kocisko, D. A. et al. “Cell-free formation of protease-resistant prion protein” Nature 370, 471-474 (1994).
Kurt, T. D. et al. “Efficient in vitro amplification of chronic wasting disease PrPres,” J. Virol. 81, 9605-9608 (2007).
LaFauci, G. et al. “Passage of chronic wasting disease prion into transgenic mice expressing Rocky Mountain elk (Cervus elaphus nelsoni) PrPC,: J. Gen. Virol. 87, 3773-3780 (2006).
Llewelyn, C. A. et al. “Possible transmission of variant CJD disease by blood transfusion,” Lancet 363, 417-421 (2004).
Madec, J-Yk. et al. “Abnormal prion protein in genetically resistant sheep from a scrapie-infected flock,” J. Gen. Virol. 85, 3483-3486 (2004).
Mathiason, C. K. et al., “Infectious prions in the saliva and blood of deer with chronic wasting disease,” Science 314, 133-136 (2006).
Mathiason, C. K. et al., “Infectious prions in pre-clinical deer and transmission of chronic wasting disease solely by environmental exposure,” PLoS One 4, e5916 (2009).
Murayama, Y. et al., “Urinary excretion and blood level of prions in scrapie-infected hamsters,” J. Gen. Virol. 88, 2890-2898 (2007).
Peden, A. H. et al. “Preclinical vCJD after blood transfusion in a PRNP codon 129 heterozygous patient” Lancet 364, 527-529 (2004).
Prusiner, S. B. et al., “Transgenetic studies implicate interactions between homologous PrP isoforms in scrapie prion replication,” Cell 63, 673-686 (1990).
Raymond, G. J. et al. “Evidence of a molecular barrier limiting susceptibility of humans, cattle, and sheep to chronic wasting disease” EMBO 19, 4425-4430 (2000).
Rubenstein R. et al. “Concentration and distribution of infectivity and PrPSc following partial denaturation of a mouse-adapted and a hamster-adapted scrapie strain.” Arch. Virol. 139, 301-311 (1994).
Saa, P. et al., “Presymptomatic detection of prions in blood,” Science 313, 92-94 (2006).
Saborio, G. P. et al., ‘Sensitive detection of pathological prion protein by cyclic amplification of protein misfolding,” Nature 411, 810-813 (2001).
Soto, C. et al., “Pre-symptomatic detection of prions by cyclic amplification of protein misfolding,” FEBS Lett. 579, 638-642 (2005).
Tamguney, G. et al., “Asymptomatic deer excrete infectious prions in faeces,” Nature 461, 529-532 (2009).
Tayebi. M. et al. “Disease-associated prion protein elicits immunoglobulin M responses in vivo,” Mol. Med. 10, 104-111 (2004).
Thorne, L. & Terry, L. A., “In vitro amplification of PrPSc derived from the brain and blood of sheep infected with scrapie,” J. Gen. Virol. 89, 3177-3184 (2008).
van Keulen, L. J., et al. “Immunohistochemical detection of prion protein in lymphoid tissues of sheep with natural scrapie,” J. Clin. Microbiol. 34, 1228-1231 (1996).
van Keulen, L. J., et al. “Immunohistochemical detection and localization of prion protein in brain tissue of sheep with natural scrapie,” Vet. Pathol. 32, 299-308 (1995).
Wells, G. A. and Wilesmith, J. W., “The neuropathology and epidemiology of bovine spongiform encephalopathy,” Brain Pathol. 5, 91-103 (1995).
Wild, M. A. and Miller, M. W., “Bottle-raising wild ruminants in captivity,” Colorado Division of Wildlife, Outdoor Facts 114 (1991).
Wild, M. A. et al, “Comparison of growth rate and milk intake of bottle-raised and dam-raised bighorn sheep, pronghorn antelope and elk neonates” J. Wildlife Management 58, 340-347 (1994).
Wolfe, L. L. et al. “PrPCWD in rectal lymphoid tissue of deer (Odocoileus spp),” J. Gen. Virol. 88, 2078-2082 (2007).

What is claimed:

1. A method for detection of the presence or absence of PrP^{Sc} in a biological sample suspected of having them comprising;
a. concentrating PrP^{Sc} as may be present in the sample by substantially separating the PrP^{Sc} from sample matrix;
b. labeling concentrated PrP^{Sc} with at least one molecular label to produce labeled PrP^{Sc}; and
c. detecting the labeled PrP^{Sc} on an instrument capable of detecting an attomole quantity of labeled PrP^{Sc}.

2. The method of claim 1, wherein the PrP^{Sc} is undigested.

3. The method of claim 1, wherein the duration of time between concentrating the PrP^{Sc} and detecting the labeled PrP^{Sc} is 48 hours or less.

4. The method of claim 1, wherein the duration of time between concentrating the PrP^{Sc} and detecting the labeled PrP^{Sc} is 24 hours or less.

5. The method of claim 1, wherein the sample comprises brain tissue, nerve tissue, blood, urine, lymphatic fluid, cerebrospinal fluid, or a combination thereof.

6. The method of claim 1, wherein the sample comprises from about 0.1 attomole to about 200 attomole of labeled PrP^{Sc}.

7. The method of claim 1, wherein the sample is not subjected to seeded polymerization.

8. The method of claim 1, wherein the molecular label is fluorescent label, phosphorescent label, radioisotope label, or a combination thereof.

9. The method of claim 8, wherein the molecular label is a fluorescent-labeled anti-PrP antibody.

10. The method of claim 9, wherein the molecular label further comprises a biotinylated anti-PrP antibody.

11. The method of claim 1, wherein the step of concentrating the PrP^{Sc} employs antibodies, immunoprecipitation, magnetic beads, or a combination thereof.

12. A method for detection of the presence or absence of PrP^{Sc} in a biological sample suspected of having them comprising;
a. amplifying PrP^{Sc} present in the sample by sPMCA;
b. concentrating PrP^{Sc} as may be present in the sample by substantially separating the PrP^{Sc} from sample matrix;
c. labeling concentrated PrP^{Sc} with at least one molecular label to produce labeled PrP^{Sc}; and
d. detecting the labeled PrP^{Sc} on an instrument capable of detecting attomole quantities of labeled PrP^{Sc}.

13. The method of claim 12, wherein the step of concentrating the PrP^{Sc} employs molecular antibodies, immunoprecipitation, magnetic beads, or a combination thereof.

14. The method of claim 12, wherein the PrP^{Sc} are undigested.

15. The method of claim 12, wherein the duration of time between amplifying PrP^{Sc} and detecting the labeled PrP^{Sc} is 48 hours or less.

16. The method of claim 12, wherein the duration of time between amplifying PrP^{Sc} and detecting the labeled PrP^{Sc} is 24 hours or less.

17. The method of claim 12, wherein the sample comprises brain tissue, nerve tissue, blood, urine, lymphatic fluid, cerebrospinal fluid, or a combination thereof.

18. The method of claim 12, wherein the sample comprises from about 0.1 attomole to about 200 attomole of labeled PrP^{Sc}.

19. The method of claim 12, wherein the molecular label is a fluorescent label, phosphorescent label, radioisotope label, or a combination thereof.

20. The method of claim 12, wherein the step of concentrating the PrP^{Sc} occurs by the monoclonal antibody or an antigen-binding portion thereof, wherein said antibody has the heavy and light chain amino acid sequences substantially identical to the antibody produced by hybridoma 08-1/8E9

21. The method of claim 12, wherein the step of labeling the PrP^{Sc} occurs by
a. monoclonal antibody or an antigen-binding portion thereof, wherein said antibody has the heavy and light chain amino acid sequences substantially identical to the antibody produced by hybridoma 08-1/11F12;
b. labelling the captured PrP^{Sc} with a biotinylated monoclonal antibody or an antigen-binding portion thereof, wherein said antibody has the heavy and light chain amino acid sequences substantially identical to the antibody produced by hybridoma 08-1/5D6.

22. The method of claim 1, wherein the analytical instrumentation is disclosed in U.S. Provisional Patent Application 61/211,264.

23. The method of claim 1, wherein the analytical instrumentation is disclosed in U.S. patent application Ser. No. 11/634,546.

24. The method of claim 12, wherein the analytical instrumentation is disclosed in U.S. Provisional Patent Application 61/211,264.

25. The method of claim 12, wherein the analytical instrumentation is disclosed in U.S. patent application Ser. No. 11/634,546.

26. A monoclonal antibody or an antigen-binding portion thereof, wherein said antibody has the heavy and light chain amino acid sequences substantially identical to the antibody produced by hybridoma 08-1/11F12.

27. A monoclonal antibody or an antigen-binding portion thereof, wherein said antibody has the heavy and light chain amino acid sequences substantially identical to the antibody produced by hybridoma 08-1/8E9

28. A monoclonal antibody or an antigen-binding portion thereof, wherein said antibody has the heavy and light chain amino acid sequences substantially identical to the antibody produced by hybridoma 08-1/5D6

29. A monoclonal antibody or antigen-binding portion thereof, which binds to PrP^{Sc} and which enhances binding of a second monoclonal antibody to PrP^{Sc}.

30. A monoclonal antibody or antigen-binding portion thereof, which binds to PrP^{Sc} in an enhanced manner after binding of a second monoclonal antibody to PrP^{Sc}.

31. A monoclonal antibody or antigen portion thereof, which normally binds to PrP^{Sc}, which cannot bind after binding of a second monoclonal antibody to PrP^{Sc}.

32. A kit for the detection of PrP^{Sc} comprising;
a. a first monoclonal antibody or antigen-binding portion thereof, which binds to PrP^{Sc} and which enhances binding of a second monoclonal antibody to PrP^{Sc}; and
b. a second monoclonal antibody or antigen-binding portion thereof, which binds to PrP^{Sc} in an enhanced manner after binding of a first monoclonal antibody to PrP^{Sc}.

33. The kit of claim 32, wherein said first antibody has the heavy and light chain amino acid sequences substantially identical to the antibody produced by hybridoma 08-1/11F12 and said second antibody has the heavy and light chain amino acid sequences substantially identical to the antibody produced by hybridoma 08-1/5D6.

34. The kit of claim 32 for the detection of PrP^{Sc} further comprising;
a third monoclonal antibody capable of immunoprecipitating PrP^{Sc}.

35. The kit of claim 32 for the detection of PrP^{Sc} further comprising;
at least one vial, cuvette or capillary for cooperation with an instrument capable of detecting attomole quantities of labeled PrP^{Sc}.
